(19) United States
(12) Patent Application Publication
(10) Pub. No.: US 2026/0347367 A1
(43) Pub. Date: Nov. 5, 2026

(54) PROCESS FOR PREPARING AN IMIDAZO[1,2-B]PYRIDAZINE

(71) Applicant: CARDURION PHARMACEUTICALS, INC., Burlington, MA (US)

(72) Inventors: John Amedio, Burlington, MA (US); Ramanaiah C. Kanamarlapudi, Burlington, MA (US); Nobuyuki Matsunaga, Fujisawa (JP); Yasufumi Miyamoto, Fujisawa (JP); Junya Shirai, Fujisawa (JP)

(21) Appl. No.: 19/653,524

(22) Filed: Apr. 21, 2026

Related U.S. Application Data
(60) Provisional application No. 63/792,719, filed on Apr. 22, 2025.

(51) Int. Cl. C07D 487/04; B01J 27/122; B01J 31/22; B01J 31/24; C07C 233/18; C07C 271/16; C07F 5/02

(52) U.S. Cl. CPC C07D 487/04; B01J 27/122; B01J 31/2282; B01J 31/2295; B01J 31/2409; C07C 233/18; C07C 271/16; C07F 5/025; B01J 2231/34; B01J 2531/004; B01J 2531/0205; B01J 2531/824; B01J 2531/842

(57) ABSTRACT

The present application generally relates to processes and intermediates for the preparation of (S)-2-(6-(3-((1-(1H-tetrazol-1-yl)propan-2-yl)oxy)-4-fluorophenyl)imidazo[1,2-b]pyridazin-3-yl)-4-methoxynicotinonitrile.

### CROSS-REFERENCE TO RELATED APPLICATION

This application claims priority to U.S. Provisional Application No. 63/792,719, filed Apr. 22, 2025, which is incorporated herein in its entirety for all purposes.

### BACKGROUND OF THE INVENTION

Cardiac diseases include heart failure, arrhythmia, myocardial infarction, angina, valvular heart disease and the like, and they are high-mortality diseases. In the treatment of cardiac diseases with a drug, the symptoms are improved by control of each risk factor and symptomatic therapy. However, satisfaction with current treatments remains at a low level, and there is no definitive therapy.

Calcium-calmodulin complex binds to Ca²⁺/calmodulin-dependent protein kinase (CaMK) included in serine/threonine protein kinase, and activates the kinase. The CaMK family includes CaMKII, and four isoforms (α, β, γ and δ) exist as CaMKII. CaMKII α and CaMKII β are expressed mainly in cerebral tissue, and CaMKII γ and CaMKII 6 are expressed in many tissues including heart. CaMKII is activated by amino acid-modification due to oxidative stress or hyperglycemia, in addition to the binding of calcium-calmodulin complex. CaMKII regulates cell functions by phosphorylating and regulating a number of cellular pathways that influence intracellular Ca²⁺ concentration homeostasis, excitation-contraction coupling, gene expression, inflammation, metabolism, and cell death.

Research suggests that CaMKII plays a harmful role in the progress of cardiac disease conditions. See, e.g., Reyes Gaido, O. E. et al. Annual Review of Pharmacology and Toxicology 2023 Jan. 20: 63: 249-272. Expression and activity of CaMKII are increased in the heart of a human patient or animal with heart failure. In transgenic mice overexpressing CaMKII 6 in the heart, onsets of cardiac hypertrophy and heart failure are reported. By studies using an inhibitor by a pharmacological method, and studies using a gene deletion by genetic method, protecting effects on heart failure, cardiac hypertrophy, myocardial infarction and arrhythmia by an inhibition of CaMKII and an overexpression of CaMKII inhibitory protein are reported in mice. For catecholaminergic polymorphic ventricular tachycardia, improving effects on disease conditions by CaMKII inhibitor in mutant ryanodine knock-in mouse (RyR2^{R4496C+/-} mouse) are reported. These findings suggest availabilities of CaMKII inhibitors in the prophylaxis and/or treatment of cardiac diseases including heart failure, cardiac hypertrophy, myocardial infarction and cardiac arrhythmia.

CaMKII exacerbating action on growth or metastasis of a certain type of cancer has been suggested. In addition, therapeutic effects on acute renal failure, intimal hypertrophy, hepatic fibrosis, stroke, pain, rheumatoid arthritis and the like by CaMKII inhibition are also indicated.

Genetic methods demonstrate only deficiency of protein or overexpression of inhibitory protein. Since these methods are different from a mechanism which temporarily inhibits kinase activity, effects by kinase inhibition in a pharmacological manner may not be predictable. In addition, inhibitors which have been reported may not be suitable for application as a medicament for a CaMKII selective inhibitor, because they have a low kinase selectivity to CaMKII, or they may not be suitable for oral administration or chronic administration.

(S)-2-(6-(3-((1-(1H-tetrazol-1-yl)propan-2-yl)oxy)-4-fluorophenyl)imidazo[1,2-b]pyridazin-3-yl)-4-methoxynicotinonitrile has been described as a CaMKII inhibitor as Example 321 (IC₅₀<10 nM) in US publication no. 20220098207 on pages 74 and 96.

US Publication no. 20220098207 describes a multistep synthesis process for preparing Compound (I). The process includes eight linear steps, containing three conversions that each result in about a 40% yield or lower from the closest intermediate. Three of the eight steps include palladium-mediated coupling.

There are difficulties associated with the adaptation of the multistep synthesis disclosed in US publication no. 20220098207 to a larger scale synthesis, such as production in a pilot plant or on a manufacturing scale. For instance, there is a continuing need to find a process that provides higher yields in order to improve manufacturing economics and/or reduce waste. Preferably, a new process would employ less expensive starting materials.

Desired is a process that is suitable for preparing larger quantities of Compound (I) than is typically prepared by laboratory scale processes. Also desired is a process that provides higher yields of Compound (I) than the previously disclosed processes.

The present invention is directed to one or both of these, as well as other important aspects.

### BRIEF SUMMARY OF THE INVENTION

In one embodiment, the present invention provides a process for preparing Compound (I):

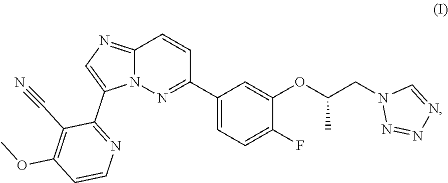 [SMILES: COc1ccnc(-c2cnc3ccc(-c4ccc(F)c(O[C@@H](C)Cn5cnnn5)c4)nn23)c1C#N]

the process comprising:
(a) reacting Compound 10:

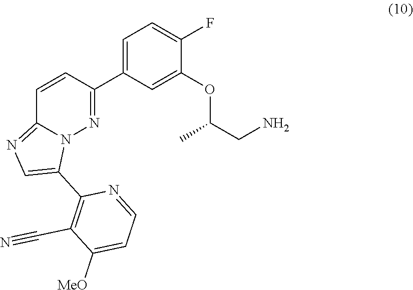 [SMILES: COc1ccnc(-c2cnc3ccc(-c4ccc(F)c(O[C@@H](C)CN)c4)nn23)c1C#N]

and a formylating agent under suitable conditions to prepare Compound 11:

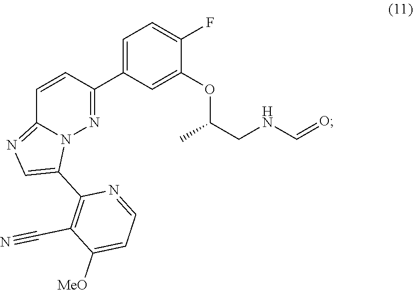 [SMILES: COc1ccnc(-c2cnc3ccc(-c4ccc(F)c(O[C@@H](C)CNC=O)c4)nn23)c1C#N]

(b) reacting Compound 11 and a dehydrating agent under suitable conditions to prepare Compound 12:

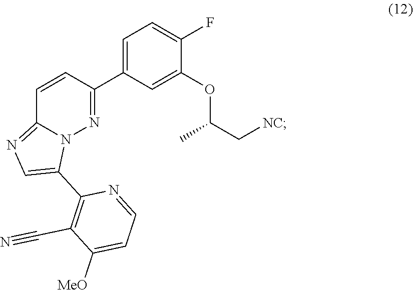

and
(c) reacting Compound 12 and an azide reagent under suitable conditions to prepare Compound (I).

In another embodiment, the present invention provides a process for preparing Compound (I):

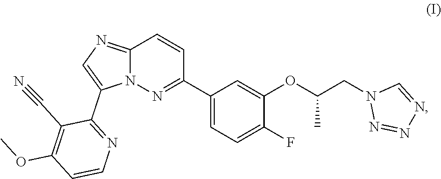 [SMILES: COc1ccnc(-c2cnc3ccc(-c4ccc(F)c(O[C@@H](C)Cn5cnnn5)c4)nn23)c1C#N]

the process comprising:
(a) reacting Compound 8:

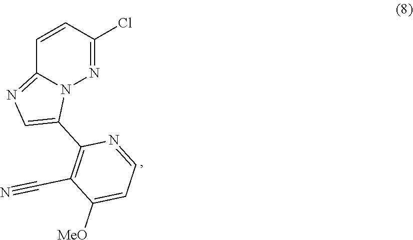 [SMILES: COc1ccnc(-c2cnc3ccc(Cl)nn23)c1C#N]

and Compound 5-Formyl:

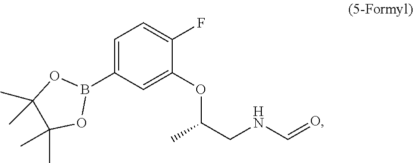 [SMILES: C[C@@H](CNC=O)Oc1cc(B2OC(C)(C)C(C)(C)O2)ccc1F]

under suitable conditions to prepare Compound 11:

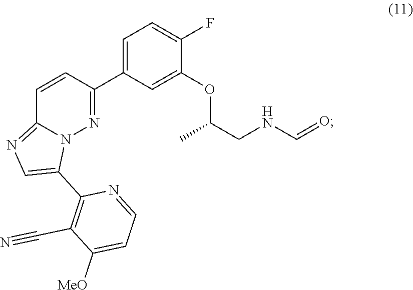 [SMILES: COc1ccnc(-c2cnc3ccc(-c4ccc(F)c(O[C@@H](C)CNC=O)c4)nn23)c1C#N]

(b) reacting Compound 11 and a dehydrating agent under suitable conditions to prepare Compound 12:

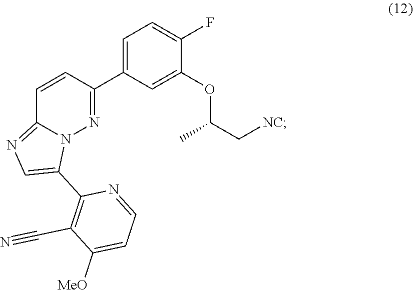

(c) reacting Compound 12 and an azide reagent under suitable conditions to prepare Compound (I).

In a further embodiment, the present invention provides a process for preparing Compound (I):

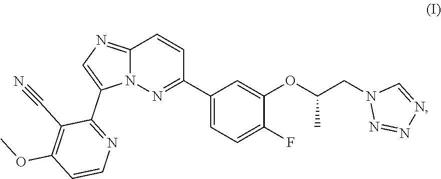 [SMILES: COc1ccnc(-c2cnc3ccc(-c4ccc(F)c(O[C@@H](C)Cn5cnnn5)c4)nn23)c1C#N]

the process comprising reacting Compound 10:

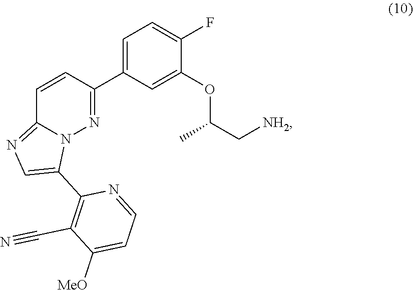 [SMILES: COc1ccnc(-c2cnc3ccc(-c4ccc(F)c(O[C@@H](C)CN)c4)nn23)c1C#N]

an azide reagent, and a formylating agent, under suitable conditions to prepare Compound (I).

In some embodiments, the present invention provides a compound having the structure of Compound Ia:

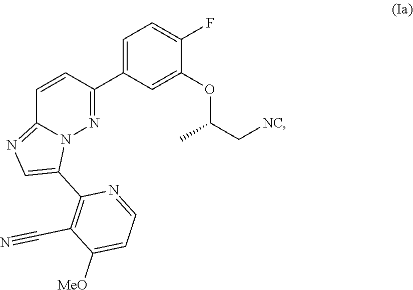

or a salt thereof.

In some embodiments, the present invention provides a compound of Formula III:

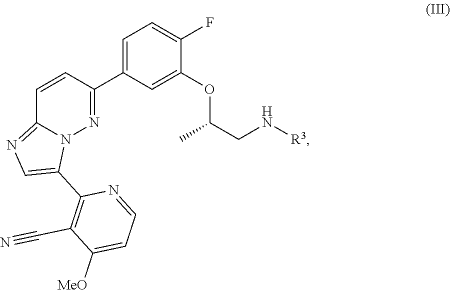

or a salt thereof, wherein

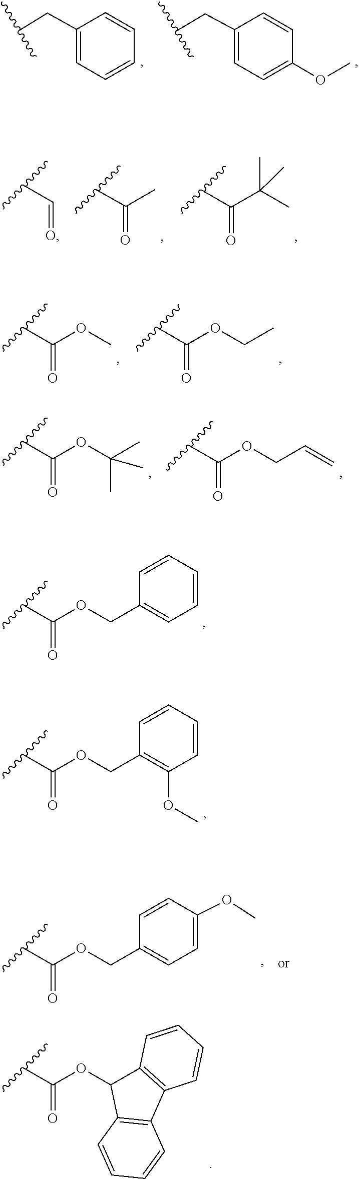

In some embodiments, the present invention provides a compound of Formula IV:

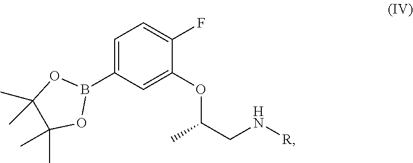

or a salt thereof,
wherein R is

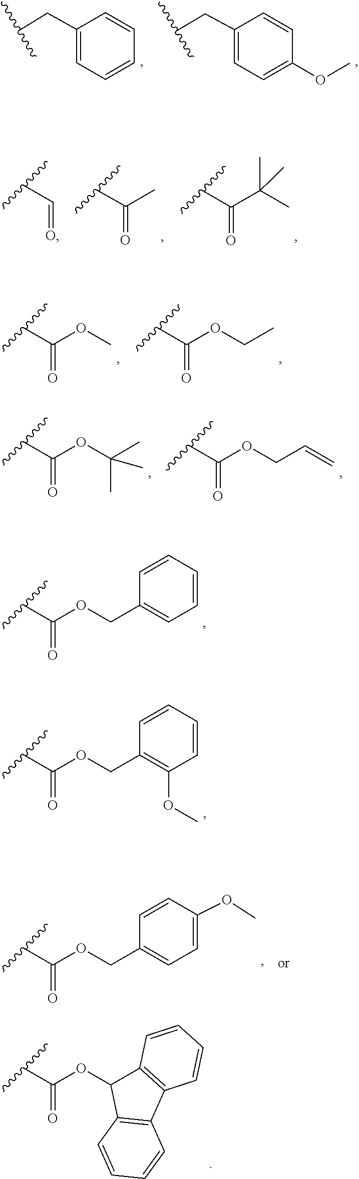

In some embodiments, the present invention provides a compound of Formula V:

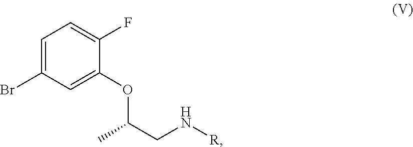

or a salt thereof,
wherein R is

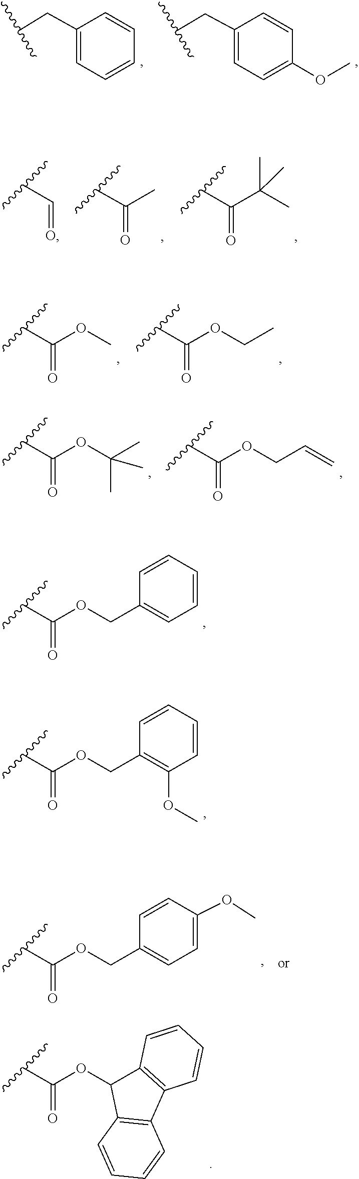

### BRIEF DESCRIPTION OF THE DRAWINGS

FIG. 1 shows the flow diagram for the synthesis of Compound (I) according to Example 2.

FIG. 2 shows the flow diagram for the synthesis of Compound (I) according to Example 3.

### DETAILED DESCRIPTION OF THE INVENTION

### I. General

The compound (S)-2-(6-(3-((1-(1H-tetrazol-1-yl)propan-2-yl)oxy)-4-fluorophenyl)imidazo[1,2-b]pyridazin-3-yl)-4-methoxynicotinonitrile (Compound I) is a selective and potent inhibitor of calcium-calmodulin (CaM)-dependent protein kinase II (CaMKII):

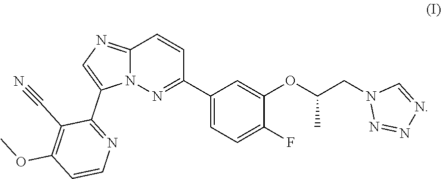 [SMILES: COc1ccnc(-c2cnc3ccc(-c4ccc(F)c(O[C@@H](C)Cn5cnnn5)c4)nn23)c1C#N]

The present disclosure describes new processes for preparing Compound (I) and new intermediates, such as Compounds 9, 10, 11, and 12, in its preparation. The processes can be performed at least on a kilogram scale and/or require fewer synthetic steps or fewer manipulations to reduce manufacturing cost or time. Example 1 shows an illustrative synthesis of Compound (I) using batch chemistry. As shown in Examples 2 and 3, certain processes may be performed in a flow reactor. For instance, tetrazole formation of Compound (I) was conducted using either batch chemistry or flow chemistry. All of the exemplary embodiments of the present disclosure demonstrated improvement compared to the known process, thereby facilitating large scale production of Compound (I).

### II. Definitions

Unless defined otherwise, all technical and scientific terms used herein have the same meaning as commonly understood by one of ordinary skill in the art. A dash at the front or end of a chemical group is a matter of convenience to indicate the point of attachment to a parent moiety; chemical groups may be depicted with or without one or more dashes without losing their ordinary meaning.

As used herein, “salts” refer to derivatives of the disclosed compounds wherein the parent compound is modified by making acid or base salts thereof. Both the free form and the salts of these compounds are within the scope of the invention. A salt can be derived from any Brønsted acid or base known in the art. Non-limiting examples of salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates.

Unless otherwise indicated, all chiral (enantiomeric and diastereomeric) and racemic forms are within the scope of the invention. Many geometric isomers of C═C double bonds, C═N double bonds, ring systems, and the like can also be present in the compounds, and all such stable isomers are contemplated in the present invention. Cis- and trans- (or E- and Z-) geometric isomers of the compounds of the present invention are described and may be isolated as a mixture of isomers or as separated isomeric forms. The present compounds can be isolated in optically active or racemic forms. Optically active forms may be prepared by resolution of racemic forms or by synthesis from optically active starting materials.

The term “stereoisomer” refers to isomers of identical constitution that differ in the arrangement of their atoms in space. Enantiomers and diastereomers are examples of stereoisomers. The term “enantiomer” refers to one of a pair of molecular species that are mirror images of each other and are not superimposable. The term “diastereomer” refers to stereoisomers that are not mirror images. The term “racemate” or “racemic mixture” refers to a composition composed of equimolar quantities of two enantiomeric species, wherein the composition is devoid of optical activity.

The symbols “R” and “S” represent the configuration of substituents around a chiral carbon atom(s). The isomeric descriptors “R” and “S” are used as described herein for indicating atom configuration(s) relative to a core molecule and are intended to be used as defined in the literature (IUPAC Recommendations 1996, Pure and Applied Chemistry, 68:2193-2222 (1996)).

The term “chiral” refers to the structural characteristic of a molecule that makes it impossible to superimpose it on its mirror image. The term “homochiral” refers to a state of enantiomeric purity. The term “optical activity” refers to the degree to which a homochiral molecule or nonracemic mixture of chiral molecules rotates a plane of polarized light.

Compounds of the present invention, free form and salts thereof, may exist in multiple tautomeric forms, in which hydrogen atoms are transposed to other parts of the molecules and the chemical bonds between the atoms of the molecules are consequently rearranged. It should be understood that all tautomeric forms, insofar as they may exist, are included within the invention.

“Alkyl” is a monovalent or divalent linear or branched saturated hydrocarbon radical. For example, an alkyl group can have 1 to 10 carbon atoms (i.e., C₁₋₁₀ alkyl) or 1 to 8 carbon atoms (i.e., C₁₋₈ alkyl) or 1 to 6 carbon atoms (i.e., C₁₋₆ alkyl) or 1 to 4 carbon atoms (i.e., C₁₋₄ alkyl). Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, and octyl (—(CH₂)₇CH₃). Alkyl groups can be unsubstituted or substituted.

The term “azide reagent” refers to any substance that serves as a source of azide (N₃⁻). An azide reagent can be inorganic, e.g., sodium azide, potassium azide, or organic, e.g., tert-butyl azide, trimethylsilyl azide, or tert-butyltrimethylsilyl azide.

A “base” includes any Lewis base and Brønsted base known in the art. Lewis bases include organic bases such as amine bases, e.g., ammonia, diisopropylethylamine (DIEA), triethylamine (TEA), tributylamine, pyridine, 2,6-lutidine, 2,2,6,6-tetramethylguanidine, 8-diazabicyclo[5.4.0]-undec-γ-ene (DBU), 1,5-diazabicyclo[4.3.0]non-5-ene (DBN), 1,4-diazabicyclo[2.2.2]octane (DABCO), and the like. A Brønsted base can be any inorganic base, such as a carbonate base or a hydroxide. Suitable hydroxide bases include, but are not limited to, potassium hydroxide, sodium hydroxide, lithium hydroxide, and the like. Suitable carbonate bases include, but are not limited to, potassium carbonate, sodium bicarbonate, sodium carbonate, lithium carbonate, and the like.

The term “formylating agent” refers to any substance that adds a formyl group (“—C(═O)H”) to an amine. Exemplary formylating agents include methyl formate and a base, chloral, formic acid, acetic formic anhydride, ammonium formate, triethyl orthoformate, trimethyl orthoformate, and the like.

The term “dehydrating agent” refers to any substance that dries or removes water from a material. In chemical reactions where dehydration occurs, the reacting molecule loses a molecule of water. A dehydrating agent can also refer to any substance that drives a dehydration reaction. Examples include, but are not limited to: phosphorus oxychloride (POCl₃), (COCl)₂, PCl₅, SOCl₂, PCl₃, dimethylchloroformiminium chloride (ClCH═N(CH₃)₂Cl, N,N′-dicyclohexylcarbodiimide (DCC), 1-ethyl-3-(3-dimethylaminopropyl)carbodiimide (EDC), N,N′-diisopropylcarbodiimide (DIC), 1-cyclohexyl-(2-morpholinoethyl)carbodiimide metho-p-toluene sulfonate (CMCT), methanesulfonyl chloride (MsCl) or 4-toluenesulfonyl chloride (TsCl) with organic bases, TiCl₄ with organic bases, PPh₃ with CCl₄ or CBr₄, SO₃·Py, pivaloyl chloride with organic bases, cyanuric chloride with organic bases, iodine and triphenylphosphine, phosphorus oxychloride and a base, 4-toluenesulfonyl chloride and a base, methanesulfonyl chloride and a base, methanesulfonic anhydride and a base, trifluoromethanesulfonic anhydride and a base, or 1,3-benzenedisulfonyl dichloride and a base.

The term “chlorinating agent” refers to any substance which adds a chlorine atom to a substrate. A chlorinating agent is a specific type of oxidizing agent. Examples include, but are not limited to: 1,3-dichloro-5,5-dimethylhydantoin, N-chlorosuccinimide (NCS), NaClO, trichloroisocyanuric acid, phosphorus oxychloride, chlorine, thionyl chloride (SOCl₂), oxalyl chloride ((COCl)₂).

The term “brominating agent” refers to any substance which adds a bromine atom to a substrate. A brominating agent is a specific type of oxidizing agent. Examples include, but are not limited to: 1,3-dibromo-5,5-dimethylhydantoin, N-bromosuccinimide (NBS), NaBrO, tribromoisocyanuric acid, phosphorus oxybromide, bromine, thionyl bromide (SOBr₂), oxalyl bromide ((COBr)₂).

### III. Processes

The present disclosure describes, inter alia, processes for preparing Compound (I) as well as processes for preparing intermediate Compound (Ia) and compounds of Formula (III), (IV), and (V).

Provided herein is a process for preparing Compound (I):

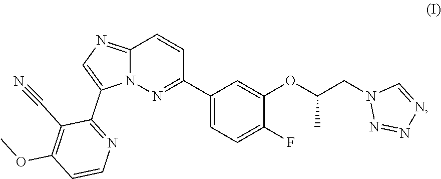 [SMILES: COc1ccnc(-c2cnc3ccc(-c4ccc(F)c(O[C@@H](C)Cn5cnnn5)c4)nn23)c1C#N]

the process comprising:
(a) reacting Compound 10:

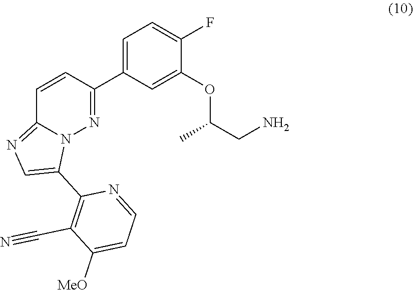 [SMILES: COc1ccnc(-c2cnc3ccc(-c4ccc(F)c(O[C@@H](C)CN)c4)nn23)c1C#N]

and a formylating agent under suitable conditions to prepare Compound 11:

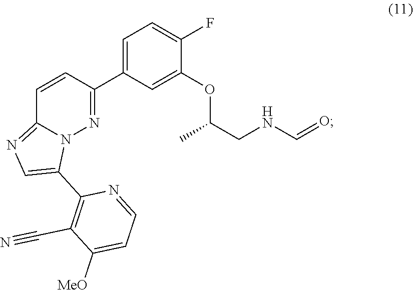 [SMILES: COc1ccnc(-c2cnc3ccc(-c4ccc(F)c(O[C@@H](C)CNC=O)c4)nn23)c1C#N]

(b) reacting Compound 11 and a dehydrating agent under suitable conditions to prepare Compound 12:

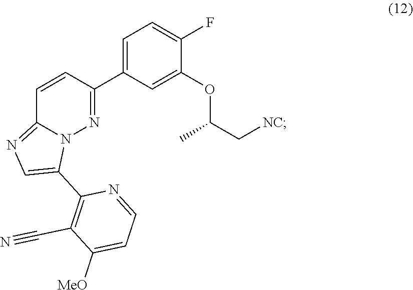

(c) reacting Compound 12 and an azide reagent under suitable conditions to prepare Compound (I).

A formylating agent used in the process of the present disclosure can be any agent capable of introducing a formyl group to an amine, e.g., a primary amine. Various methods of formylating primary amines have been reported. See, e.g., Gerack, C. J.; McElwee-White, L. Molecules 2014, vol. 19, pages 7689-7713, and references cited therein. In some embodiments, the formylating agent comprises trimethyl orthoformate, triethyl orthoformate, methyl formate and a base, chloral, formic acid, acetic formic anhydride, ammonium formate, carbon dioxide and trimethoxysilane, carbon dioxide and phenylsilane, or ethyl bromodifluoroacetate. In some embodiments, the base is potassium carbonate, lithium carbonate, or sodium carbonate. In some embodiments, the formylating agent comprises methyl formate and potassium carbonate.

The dehydrating agent used in the processes of the present disclosure can be any dehydrating agent known in the art. In some embodiments, the dehydrating agent is iodine and triphenylphosphine, phosphorus oxychloride and a base, 4-toluenesulfonyl chloride and a base, methanesulfonyl chloride and a base, methanesulfonic anhydride and a base, trifluoromethanesulfonic anhydride and a base, or 1,3-benzenedisulfonyl dichloride and a base. In some embodiments, the dehydrating agent is iodine and triphenylphosphine. In some embodiments, the dehydrating agent comprises a base.

A base used in the processes of the present disclosure, e.g., with a dehydrating agent in preparing Compound (I), can be any Lewis base or Brønsted base known in the art. In some embodiments, the base is an inorganic base. In some embodiments, the base is an organic base. In some embodiments, the base is triethylamine, diisopropylethylamine, pyridine, N-methylimidazole, DABCO, lithium bicarbonate, lithium carbonate, sodium bicarbonate, sodium carbonate, potassium bicarbonate, or potassium carbonate. In some embodiments, the base is triethylamine.

Any suitable solvent can be used in the reaction of Compound 11 to produce Compound 12. In some embodiments, the solvent is a aprotic solvent. In some embodiments, the solvent comprises dichloromethane, chloroform, or TIHF. In some embodiments, the solvent comprises dichloromethane.

In some embodiments, the process of preparing the Compound 12 comprises purifying the Compound 12. In some embodiments, the purifying the Compound 12 comprises activated carbon. In some embodiments, the purifying the Compound 12 comprises combining with an amine, such as ethylenediamine.

The azide reagent used in the processes of the present disclosure can be any azide reagent known in the art. In some embodiments, the azide reagent is a compound of Formula II:

R¹—N₃ (II),
wherein
R¹ is Na, K, tert-butyl, benzyl, o-OMe benzyl, p-OMe benzyl, p-toluenesulfonyl, or

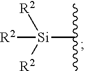

and
each R² is independently C₁₋₆ alkyl or phenyl.

In some embodiments, the azide reagent is trimethylsilyl azide.

Any suitable solvent can be used in the isocyanide reaction with an azide reagent to produce the Compound (I). In some embodiments, the solvent is a protic solvent. In some embodiments, the solvent comprises trifluoroethanol, ethanol, or water. In some embodiments, the solvent comprises trifluoroethanol.

In another embodiment, a process comprises preparing Compound 10 by deprotecting Compound 9:

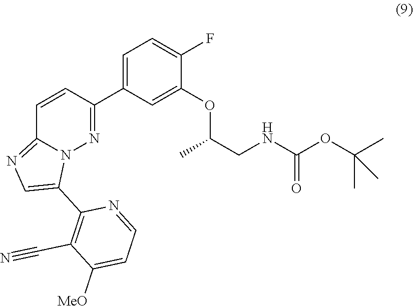 [SMILES: COc1ccnc(-c2cnc3ccc(-c4ccc(F)c(O[C@@H](C)CNC(=O)OC(C)(C)C)c4)nn23)c1C#N]

under suitable conditions to prepare Compound 10.

In some embodiments, the process further comprises preparing Compound 9 by reacting Compound 8:

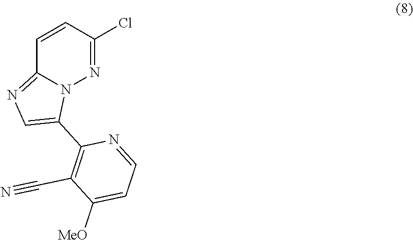 [SMILES: COc1ccnc(-c2cnc3ccc(Cl)nn23)c1C#N]

and
Compound 5-Boc:

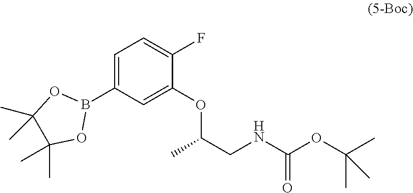 [SMILES: C[C@@H](CNC(=O)OC(C)(C)C)Oc1cc(B2OC(C)(C)C(C)(C)O2)ccc1F]

under suitable conditions to prepare Compound 9.

In some embodiments, the process further comprises preparing Compound 9 by reacting Compound 8:

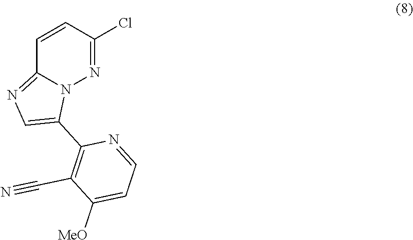 [SMILES: COc1ccnc(-c2cnc3ccc(Cl)nn23)c1C#N]

Compound 5-B(OH):

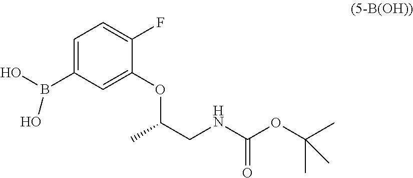 [SMILES: C[C@@H](CNC(=O)OC(C)(C)C)Oc1cc(B(O)O)ccc1F]

under suitable conditions to prepare Compound 9.

In some embodiments, the process of preparing Compound 9 comprises a palladium catalyst. Any suitable palladium(O) catalyst known in the art, or any palladium(II) salt capable of being reduced to palladium(O) under suitable reaction conditions, can be used in the processes of the present disclosure. In some embodiments, the palladium catalyst comprises tetrakis(triphenylphosphine)palladium(O), bis(triphenylphosphine)palladium(II) dichloride, palladium(II) acetate, 1,1′-bis(diphenylphosphino)ferrocene-palladium(II) dichloride, tris(dibenzylideneacetone)dipalladium(O), dichloro[9,9-dimethyl-4,5-bis(diphenylphosphino)xanthene]palladium(II), palladium(II) trifluoroacetate, or bis(acetonitrile)dichloropalladium(II). In some embodiments, the palladium catalyst comprises tetrakis(triphenylphosphine)palladium(O).

In some embodiments, the process of preparing Compound 9 comprises a copper catalyst. Any suitable copper(I) catalyst known in the art, or any copper(II) salt capable of being reduced to copper(I) under suitable reaction conditions, can be used in the processes of the present disclosure. In some embodiments, the copper catalyst comprises copper(I) iodide, copper(I) bromide, copper(I) chloride, or copper(I) trifluoromethanesulfonate. In some embodiments, the copper catalyst comprises copper(I) iodide.

Any suitable solvent known in the art can be used in the Suzuki-Miyaura coupling reaction of Compound 8 to produce Compound 9. In some embodiments, the solvent comprises a protic solvent. In some embodiments, the solvent comprises toluene, ethanol, and water.

In some embodiments, the process for preparing Compound (I) comprises purifying the Compound (I). In some embodiments, the purifying the Compound (I) comprises crystallizing the Compound (I). In some embodiments, the crystallizing the Compound (I) comprises warming Compound (I) in ethanol and water to from about 25 °C to about 100 °C, such as from about 30 °C to about 90 °C, from about 40 °C to about 90 °C, from about 50 °C to about 90 °C, from about 60 °C to about 90 °C, from about 70 °C to about 90 °C, from about 30 °C to about 80 °C, from about 40 °C to about 80 °C, from about 50 °C to about 80 °C, from about 60 °C to about 80 °C, or from about 70 °C to about 80 °C.

Also provided herein is a process for preparing Compound (I):

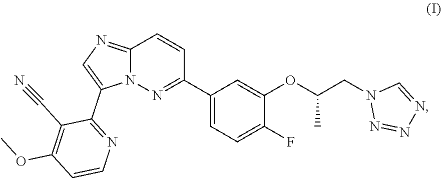 [SMILES: COc1ccnc(-c2cnc3ccc(-c4ccc(F)c(O[C@@H](C)Cn5cnnn5)c4)nn23)c1C#N]

the process comprising:
(a) reacting Compound 8:

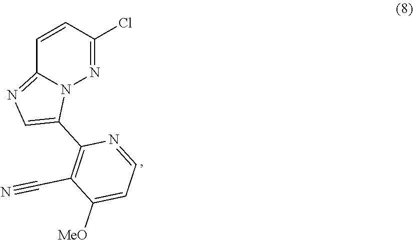 [SMILES: COc1ccnc(-c2cnc3ccc(Cl)nn23)c1C#N]

and Compound 5-Formyl:

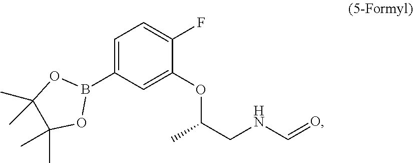 [SMILES: C[C@@H](CNC=O)Oc1cc(B2OC(C)(C)C(C)(C)O2)ccc1F]

under suitable conditions to prepare Compound 11:

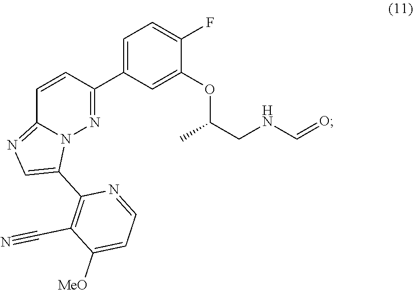 [SMILES: COc1ccnc(-c2cnc3ccc(-c4ccc(F)c(O[C@@H](C)CNC=O)c4)nn23)c1C#N]

(b) reacting Compound 11 and a dehydrating agent under suitable conditions to prepare Compound 12:

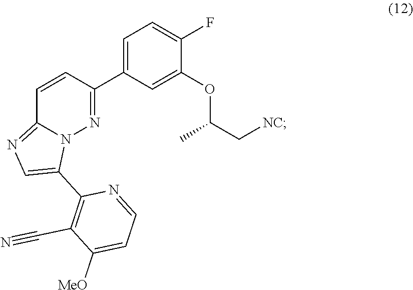

(c) reacting Compound 12 and an azide reagent under suitable conditions to prepare the Compound (I).

In some embodiments, the process of preparing Compound 11 further comprises a palladium catalyst. Any suitable palladium(O) catalyst known in the art, or any palladium(II) salt capable of being reduced to palladium(O) under suitable reaction conditions, can be used in the processes of the present disclosure. In some embodiments, the palladium catalyst comprises tetrakis(triphenylphosphine)palladium(O), bis(triphenylphosphine)palladium(II) dichloride, palladium(II) acetate, 1,1′-bis(diphenylphosphino)ferrocene-palladium(II) dichloride, tris(dibenzylideneacetone)dipalladium(O), dichloro[9,9-dimethyl-4,5-bis(diphenylphosphino)xanthene]palladium(II), palladium(II) trifluoroacetate, or bis(acetonitrile)dichloropalladium(II). In some embodiments, the palladium catalyst comprises tetrakis(triphenylphosphine)palladium(O).

In some embodiments, the process of preparing Compound 11 comprises a copper catalyst. Any suitable copper(I) catalyst known in the art, or any copper(II) salt capable of being reduced to copper(I) under suitable reaction conditions, can be used in the processes of the present disclosure. In some embodiments, the copper catalyst comprises copper(I) iodide, copper(I) bromide, copper(I) chloride, or copper(I) trifluoromethanesulfonate. In some embodiments, the copper catalyst comprises copper(I) iodide.

Any suitable solvent known in the art can be used in the Suzuki-Miyaura coupling reaction of Compound 8 to produce Compound 11. In some embodiments, the solvent comprises a protic solvent. In some embodiments, the solvent comprises toluene, ethanol, and water.

In some embodiments, the process of preparing Compound 11 comprises preparing Compound 5-Formyl by reacting Compound 4-Formyl:

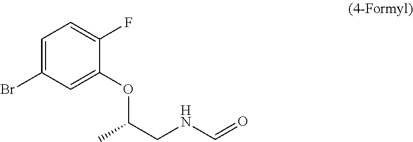

and bis(pinacolato)diboron under suitable conditions to prepare Compound 5-Formyl.

In some embodiments, the process for preparing Compound (I) comprises purifying the Compound (I). In some embodiments, the purifying the Compound (I) comprises crystallizing the Compound (I). In some embodiments, the crystallizing the Compound (I) comprises warming Compound (I) in ethanol and water to from about 25 °C to about 100 °C, such as from about 30 °C to about 90 °C, from about 40 °C to about 90 °C, from about 50 °C to about 90 °C, from about 60 °C to about 90 °C, from about 70 °C to about 90 °C, from about 30 °C to about 80 °C, from about 40 °C to about 80 °C, from about 50 °C to about 80 °C, from about 60 °C to about 80 °C, or from about 70 °C to about 80 °C.

The present disclosure also provides a process for preparing Compound (I):

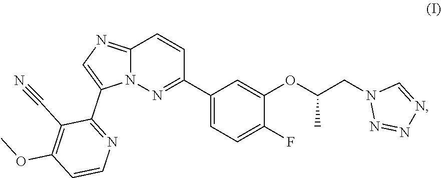 [SMILES: COc1ccnc(-c2cnc3ccc(-c4ccc(F)c(O[C@@H](C)Cn5cnnn5)c4)nn23)c1C#N]

the process comprising reacting Compound 10:

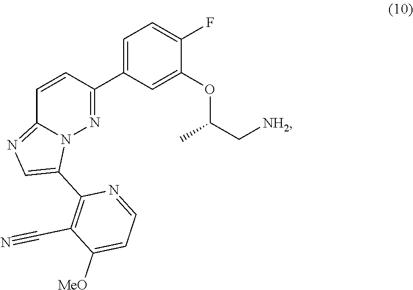 [SMILES: COc1ccnc(-c2cnc3ccc(-c4ccc(F)c(O[C@@H](C)CN)c4)nn23)c1C#N]

an azide reagent, and a formylating agent, under suitable conditions to prepare Compound (I).

The azide reagent used in the processes can be any azide reagent known in the art. In some embodiments, the azide reagent is a compound of Formula II:

R¹—N₃ (11),
wherein
R¹ is Na, K, tert-butyl, benzyl, o-OMe benzyl, p-OMe benzyl, p-toluenesulfonyl, or

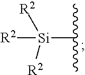

each R² is independently C₁₋₆ alkyl or phenyl.

In some embodiments, the azide reagent is trimethylsilyl azide.

In some embodiments of the process for preparing Compound (I), the formylating agent comprises trimethyl orthoformate, triethyl orthoformate, methyl formate and a base, chloral, formic acid, acetic formic anhydride, ammonium formate, carbon dioxide and trimethoxysilane, carbon dioxide and phenylsilane, or ethyl bromodifluoroacetate. In some embodiments, the formylating agent is trimethyl orthoformate.

In some embodiments, the process for preparing Compound (I) comprises purifying the Compound (I). In some embodiments, the purifying the Compound (I) comprises crystallizing the Compound (I). In some embodiments, the crystallizing the Compound (I) comprises warming Compound (I) in ethanol and water to from about 25 °C to about 100 °C, such as from about 30 °C to about 90 °C, from about 40 °C to about 90 °C, from about 50 °C to about 90 °C, from about 60 °C to about 90 °C, from about 70 °C to about 90 °C, from about 30 °C to about 80 °C, from about 40 °C to about 80 °C, from about 50 °C to about 80 °C, from about 60 °C to about 80 °C, or from about 70 °C to about 80 °C.

Further provided herein is a process of preparing Compound 8:

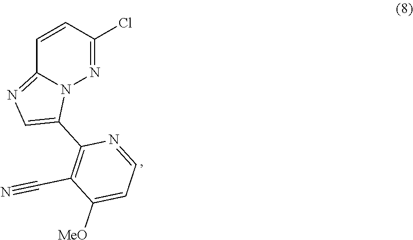 [SMILES: COc1ccnc(-c2cnc3ccc(Cl)nn23)c1C#N]

the process comprising:
(a) reacting Compound 1:

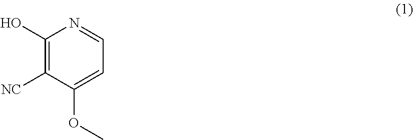

and a chlorinating agent to prepare Compound 2:

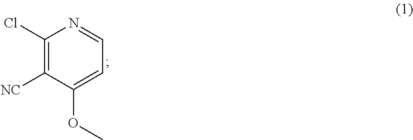

(b) reacting Compound 2 and Compound A:

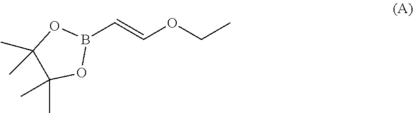

under suitable conditions to prepare Compound 3:

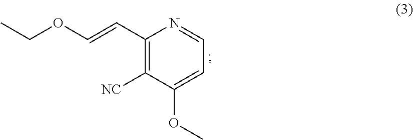

(c) reacting Compound 3, Compound B:

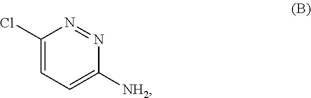

a brominating agent under suitable conditions to prepare Compound 8.

In some embodiments of the process of preparing Compound 8, the chlorinating agent comprises phosphorus oxychloride, trichloroacetonitrile, or N-chlorosuccinimide. In some embodiments, the chlorinating agent is phosphorus oxychloride.

In some embodiments of the process of preparing Compound 8, the brominating agent comprises N-bromosucccinimide, phosphorus oxybromide, or bromine. In some embodiments, the brominating agent is N-bromosucccinimide.

Suitable conditions for the reactions described herein will be understood by a skilled artisan. In some embodiments, reaction conditions such as temperature, reaction time, solvents, work-up procedures, and the like, will be those common in the art for the particular process to be performed. Typically the temperatures can be −100 °C to 200 °C, solvents can be aprotic or protic, and reaction times can be 10 seconds to 10 days. Work-up typically consists of quenching any unreacted reagents followed by partition between a water/organic layer system (extraction) and separating the layer containing the product.

The processes of the present disclosure can provide Compound (I), Compound (Ia), or a compound of Formula (III), (IV), or (V) in any suitable yield. For example, Compound (I), Compound (Ia), or a compound of Formula (III), (IV), or (V) can be prepared in a yield of at least 50%, 55%, 60%, 65%, 70%, 75%, 80%, 85%, 90%, 95%, 97%, 98%, or at least 99%. In some embodiments, the yield of Compound (I), Compound (Ia), or a compound of Formula (III), (IV), or (V) is from about 60% to about 100%. In some embodiments, the yield of Compound (I), Compound (Ia), or a compound of Formula (III), (IV), or (V) is from about 70% to about 80% or from about 75% to about 85%. In some embodiments, the yield of Compound (I), Compound (Ia), or a compound of Formula (III), (IV), or (V) is about 60%, about 70%, about 72%, about 74%, about 75%, about 76%, about 78%, about 80%, about 82%, about 84%, about 85%, about 86%, about 88%, about 90%, about 95%, about 97%, about 98%, or about 99%. In some embodiments, the yield of Compound (I), Compound (Ia), or a compound of Formula (III), (IV), or (V) is about 79%. In some embodiments, the yield of Compound (I), Compound (Ia), or a compound of Formula (III), (IV), or (V) is from about 60% to about 90%. In some embodiments, the yield of Compound (I), Compound (Ia), or a compound of Formula (III), (IV), or (V) is from about 70% to about 90%. In some embodiments, the yield of Compound (I), Compound (Ia), or a compound of Formula (III), (IV), or (V) is from about 70% to about 80%. In some embodiments, the yield of Compound (I), Compound (Ia), or a compound of Formula (III), (IV), or (V) is from about 75% to about 85%.

The processes of the present disclosure can provide Compound (I), Compound (Ia), or a compound of Formula (III), (IV), or (V) in any suitable purity. For example, Compound (I), Compound (Ia), or a compound of Formula (III), (IV), or (V) can be prepared in a purity of from about 90% to about 100%, such as from about 95% to about 100% or from about 98% to about 100%. In some embodiments, the purity of Compound (I), Compound (Ia), or a compound of Formula (III), (IV), or (V) is from about 98% to about 100%. In some embodiments, Compound (I), Compound (Ia), or a compound of Formula (III), (IV), or (V) is prepared in a purity of about 90%, about 95%, about 96%, about 97%, about 98%, about 99%, about 99.9%, about 99.99%, about 99.999%, about 99.9999%, or about 99.99999%. In some embodiments, Compound (I), Compound (Ia), or a compound of Formula (III), (IV), or (V) is prepared in a purity of about 99.9%. In some embodiments, Compound (I), Compound (Ia), or a compound of Formula (III), (IV), or (V) is prepared in a purity of from about 95% to about 99.999%, from about 98% to about 99.999%, from about 98% to about 99.99%, or from about 99% to about 99.99%. In some embodiments, the purity of Compound (I), Compound (Ia), or a compound of Formula (III), (IV), or (V) is from about 90% to about 100%.

The processes of the present disclosure can provide Compound (I), Compound (Ia), or a compound of Formula (III), (IV), or (V) in any suitable stereochemical purity, e.g., enantiomeric purity. For example, Compound (I), Compound (Ia), or a compound of Formula (III), (IV), or (V) can be prepared in an enantiomeric excess (ee) from about 90% to about 100%, such as from about 95% to about 100% or from about 98% to about 100%. In some embodiments, the enantiomeric excess of Compound (I), Compound (Ia), or a compound of Formula (III), (IV), or (V) is from about 98% to about 100%. In some embodiments, Compound (I), Compound (Ia), or a compound of Formula (III), (IV), or (V) is prepared in an enantiomeric excess of about 90%, about 95%, about 96%, about 97%, about 98%, about 99%, about 99.9%, about 99.99%, about 99.999%, about 99.9999%, or about 99.99999%. In some embodiments, Compound (I), Compound (Ia), or a compound of Formula (III), (IV), or (V) is prepared in an enantiomeric excess of about 99.9%. In some embodiments, Compound (I), Compound (Ia), or a compound of Formula (III), (IV), or (V) is prepared in an enantiomeric excess of from about 95% to about 99.999%, from about 98% to about 99.999%, from about 98% to about 99.99%, or from about 99% to about 99.99%. In some embodiments, the enantiomeric excess of Compound (I), Compound (Ia), or a compound of Formula (III), (IV), or (V) is from about 90% to about 100%.

### IV. Compounds

The present disclosure includes intermediate compounds related to the processes of the present invention.

In some embodiments, a compound of the present disclosure has the structure of Compound (Ia):

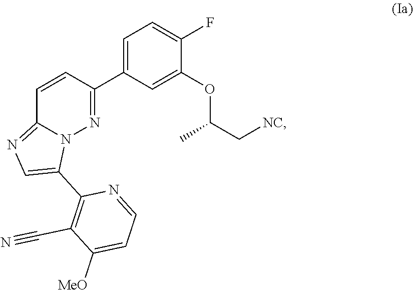

or a salt thereof.

In some embodiments, a compound of the present disclosure is compound of Formula III:

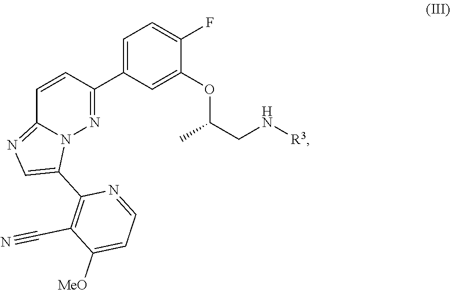

or a salt thereof, wherein
R³ is H,

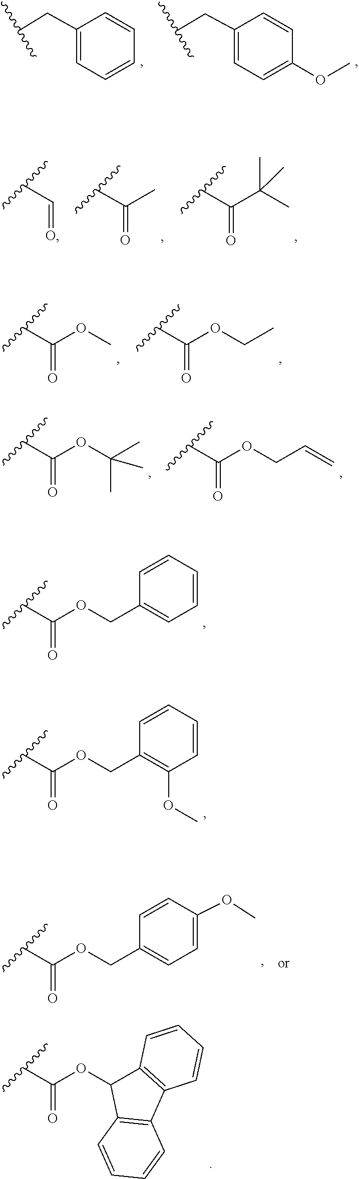

In some embodiments, R³ is H,

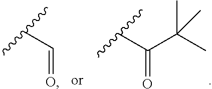

In some embodiments, the compound of Formula III has the structure:

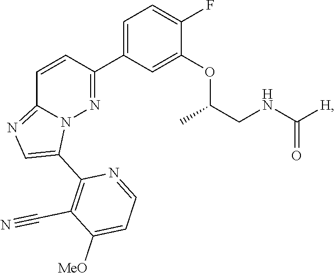

or a salt thereof.

In some embodiments, the compound of Formula III has the structure:

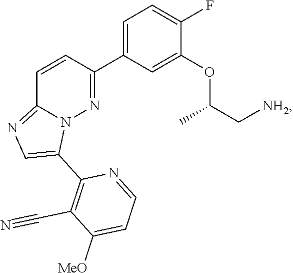 [SMILES: COc1ccnc(-c2cnc3ccc(-c4ccc(F)c(O[C@@H](C)CN)c4)nn23)c1C#N]

or a salt thereof. In some embodiments, the salt is a hydrochloride salt.

In some embodiments, the compound of Formula III has the structure:

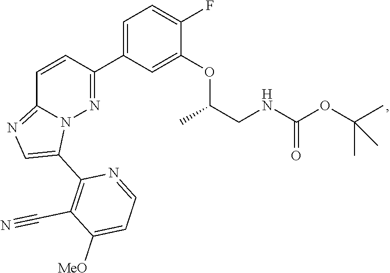 [SMILES: COc1ccnc(-c2cnc3ccc(-c4ccc(F)c(O[C@@H](C)CNC(=O)OC(C)(C)C)c4)nn23)c1C#N]

or a salt thereof.

In some embodiments, a compound of the present disclosure is a compound of Formula IV:

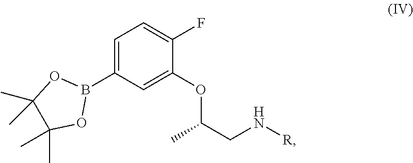

or a salt thereof,
wherein R is

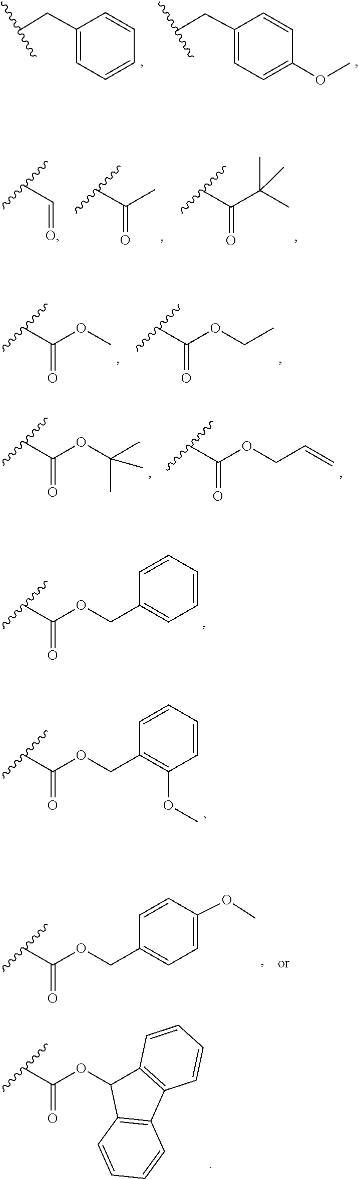

In some embodiments, R is

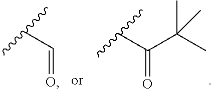

In some embodiments, a compound of Formula IV has the structure:

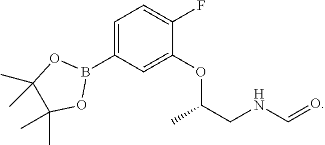 [SMILES: C[C@@H](CNC=O)Oc1cc(B2OC(C)(C)C(C)(C)O2)ccc1F]

In some embodiments, a compound of Formula IV has the structure:

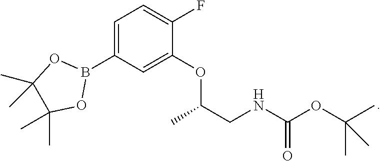 [SMILES: C[C@@H](CNC(=O)OC(C)(C)C)Oc1cc(B2OC(C)(C)C(C)(C)O2)ccc1F]

In some embodiments, a compound of the present disclosure is a compound of Formula V:

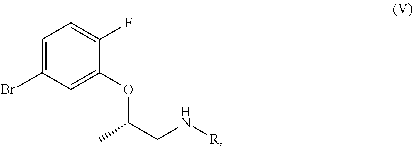

or a salt thereof,
wherein R is

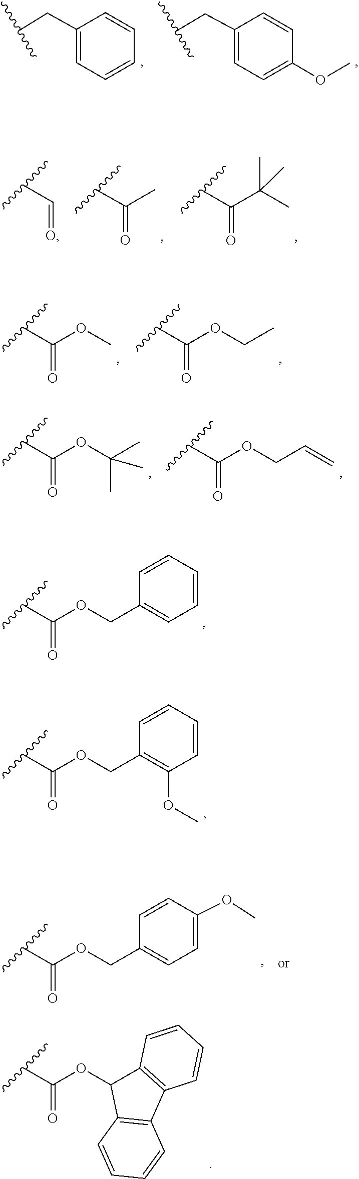

In some embodiments, R is

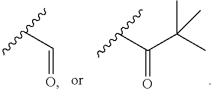

In some embodiments, a compound of Formula V has the structure:

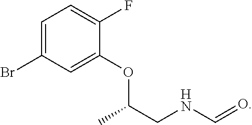

In some embodiments, a compound of Formula V has the structure:

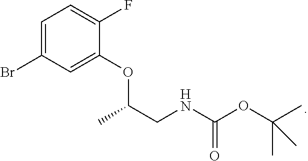 [SMILES: C[C@@H](CNC(=O)OC(C)(C)C)Oc1cc(Br)ccc1F]

Salts can be derived from inorganic or organic acids or bases. For example, a compound that contains a basic nitrogen can be prepared as a pharmaceutically acceptable salt by contacting the compound with an inorganic or organic acid. Non-limiting examples of salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable salts are found in Remington: The Science and Practice of Pharmacy, 21^{st} Edition, Lippincott Wiliams and Wilkins, Philadelphia, Pa., 2006.

Examples of salts of the compounds disclosed herein also include salts derived from an appropriate base, such as an alkali metal (for example, sodium, potassium), an alkaline earth metal (for example, magnesium), ammonium and NX₄+(wherein X is C₁-C₄ alkyl).

Also included are base addition salts, such as sodium or potassium salts.

### V. Examples

The following examples are provided to further aid in understanding the embodiments disclosed in the application, and presuppose an understanding of conventional methods well known to those persons having ordinary skill in the art to which the examples pertain. The particular materials and conditions described hereunder are intended to exemplify particular aspects of embodiments disclosed herein and should not be construed to limit the reasonable scope thereof.

Many general references providing commonly known chemical synthetic schemes and conditions useful for synthesizing the disclosed compounds are available (see, e.g., Smith, March's Advanced Organic Chemistry: Reactions, Mechanisms, and Structure, 7^{th} edition, Wiley-Interscience, 2013.)

Compounds as described herein can be purified by any of the means known in the art, including chromatographic means, such as high performance liquid chromatography (HPLC), preparative thin layer chromatography, flash column chromatography and ion exchange chromatography. Any suitable stationary phase can be used, including normal and reversed phases as well as ionic resins. For example, disclosed compounds can be purified via silica gel chromatography. See, e.g., Introduction to Modem Liquid Chromatography, 2nd ed., ed. L. R. Snyder and J. J. Kirkland, John Wiley and Sons, 1979; and Thin Layer Chromatography, E. Stahl (ed.), Springer-Verlag, New York, 1969. Compounds were characterized using standard instrumentation methods.

Identification of the compound was carried out by hydrogen nuclear magnetic resonance spectrum (¹H-NMR) and mass spectrum (MS). ¹H-NM/IR was measured at 400 MHUz, unless otherwise specified. In some cases, exchangeable hydrogen could not be clearly observed depending on the compound and measurement conditions. The designation br. or broad, used herein, refers to a broad signal. PLC preparative chromatography was carried out by a commercially available ODS column in a gradient mode using water/methanol (containing formic acid) as eluents, unless otherwise specified.

Certain abbreviations and acronyms are used in describing the experimental details. Although most of these would be understood by one skilled in the art, Table 1 contains a list of many of these abbreviations and acronyms.

TABLE 1
List of Abbreviations and Acronyms

| Abbreviation | Meaning |
|---|---|
| °C | degree(s) Celsius |
| μg or μg | microgram(s) |
| μL or μL | microliter(s) |
| μm or μm | micron(s) |
| μmol or μmol | micromole(s) |
| ACN or MeCN | acetonitrile |
| aq | aqueous |
| Boc | tert-butoxycarbonyl |
| (Bpin)₂ | bis(pinacolato)diboron |
| br s | broad singlet |
| cataCXium A | di(1-adamanty1)-n-butylphosphine |
| Cbz | benzyloxycarbonyl |
| d | doublet |
| DCM | dichloromethane |
| DIAD | diisopropyl azodicarboxylate |
| DIEA or DIPEA | N,N-diisopropylethylamine |
| DMA | dimethylacetamide |
| DMB | 2,4-dimethoxybenzyl |
| DMF | dimethylformamide |
| DMSO | dimethyl sulfoxide |
| dppf | 1,1′-bis(diphenylphosphino)ferrocene |
| Et | ethyl |
| EtOAc | ethyl acetate |
| g | gram(s) |
| h | hour(s) |
| HPLC | high-performance liquid chromatography |
| Hz | Hertz |
| iPr | isopropyl |
| IPA or iPrOH | isopropyl alcohol |
| J | coupling constant |
| KOAc | potassium acetate |
| LCMS | liquid chromatography mass spectrometry |
| m | multiplet |
| M | molarity |
| Me | methyl |
| MeOH | methanol |
| mg | milligram(s) |
| MHz | megahertz |
| min | minute(s) |
| mL | milliliter(s) |
| mm | millimeter(s) |
| mmol | millimole(s) |
| MTBE | methyl tert-butyl ether |
| NBS | N-bromosucccinimide |
| NMR | nuclear magnetic resonance |
| OAc | acetate |
| OTf | trifluoromethanesulfonate |
| qd | quartet of doublets |
| QNMR | quantitative nuclear magnetic resonance |
| rt or RT | room temperature |
| S | singlet |
| sat. | saturated |
| sec-BuLi | sec-butyllithium |
| t | triplet |
| TBAF | tetrabutylammonium fluoride |
| ^{t}Bu | tert-butyl |
| TEA | triethylamine |
| TF₂O or Tf₂O | trifluoromethanesulfonic anhydride |
| TFA | trifluoroacetic acid |
| TFE | trifluoroethanol |
| THF | Tetrahydrofuran |
| TMOF | trimethyl orthoformate |
| TMS | trimethylsilyl |
| TMSCI | trimethylsilyl chloride |
| tol | toluene |
| tt | triplet of triplets |
| v/V | volume/volume |
| wt | weight |
| Xphos | dicyclohexyl[2′,4′,6′-tris(propan-2-yl) |
| | [1,1′-biphenyl]-2-yl]phosphane |
| δ | parts per million referenced to residual |
| | non-deuterated solvent peak |

### Example 1. Compound (I) Synthesis with Batch Chemistry

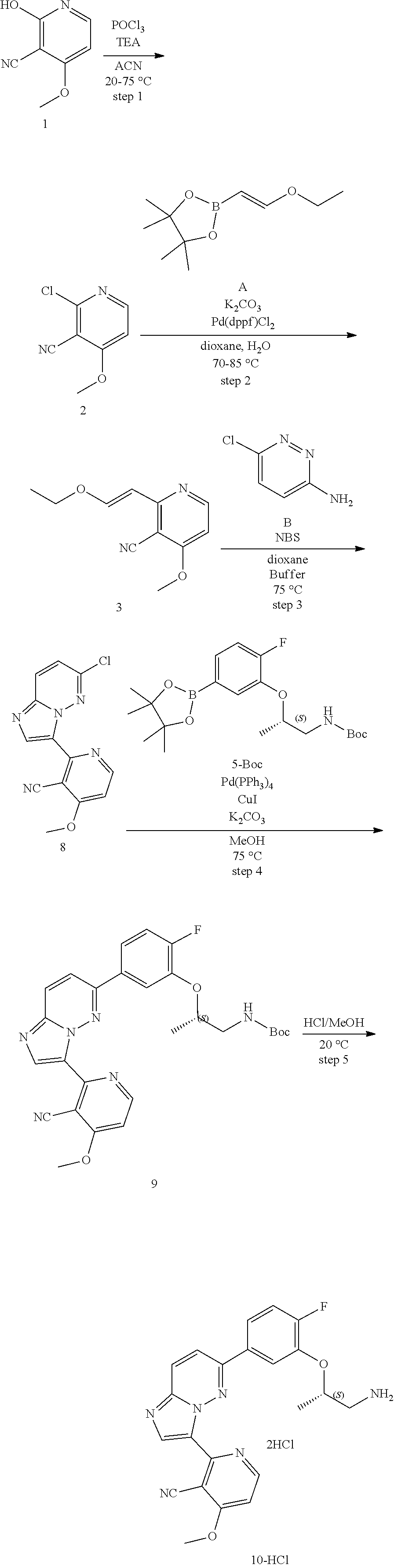

### 2-Chloro-4-methoxynicotinonitrile (2)

To a solution of 2-hydroxy-4-methoxynicotinonitrile (1) (87.0 kg) in dry MeCN (547 kg) was added TEA (41.0 kg) and POCl₃ (222 kg) at 20-30 °C under nitrogen atmosphere. The reaction mixture was stirred at 70-75 °C for 6 hrs. The reaction mixture was monitored by HPLC until starting material was consumed. The reaction mixture was dropwise added into a mixture of TEA (378 kg) in H₂O (626 kg) and dioxane (43.5 kg) at 35-45 °C to form suspension under nitrogen atmosphere. The suspension was stirred at 45-55 °C for 1 hr. Then the suspension was cooled to 0-5 °C and stirred for 1 hr. The suspension was filtered. The filter cake was washed with H₂O (522 kg) and dried at 55 °C to obtain the titled compound (87.0 kg, 516 mol, 83% yield, QNMR: 92%) a white solid: ¹H NMR (400 MHz, DMSO-d₆) δ 8.53 (d, J=6.0 Hz, 1H), 7.38 (d, J=6.4 Hz, 1H), 4.05 (s, 3H).

### (E)-2-(2-Ethoxyvinyl)-4-methoxynicotinonitrile (3)

To a solution of 2-chloro-4-methoxynicotinonitrile (2) (72.0 kg) in dioxane (595 kg) and H₂O (288 kg) were added (E)-2-(2-ethoxyvinyl)-4,4,5,5-tetramethyl-1,3,2-dioxaborolane (Compound A) (118 kg), K₂CO₃ (177 kg) and Pd(dppf)Cl₂ (9.37 kg) at 20 °C under nitrogen protection. The reaction mixture was degassed with nitrogen for 2 hrs. Then the reaction mixture was stirred at 70-85 °C for 15 hrs under nitrogen atmosphere. The reaction mixture was monitored by HPLC until starting Compound 2 was consumed. The reaction mixture was separated, the organic phase was product. The organic phase was added into a mixture of NaH₂PO₄ (126 kg) and Na₂HPO₄ (153 kg) in H₂O (2160 kg) at 15-25 °C to form a suspension. The suspension was stirred at 55-60 °C for 1 hr. Then the suspension was cooled to 30-35 °C and stir for 1 hr. The suspension was filtered. The filter cake was washed with H₂O (432 kg) and dried at 55 °C to obtain the titled compound (120 kg, 352 mol, 84% yield, QNMR: 60%) as a brown solid: ¹H NMR (400 MHz, DMSO-d₆) δ 8.46 (d, J=6.0 Hz, 1H), 7.88 (d, J=12 Hz, 1H), 6.97 (d, J=6.0 Hz, 1H), 6.00 (d, J=12 Hz, 1H), 4.01-4.07 (m, 2H), 3.95 (s, 3H), 1.29 (t, J=7.2 Hz, 3H).

### 2-(6-Chloroimidazo[1,2-b]pyridazin-3-yl)-4-methoxynicotinonitrile (8)

To a solution of Na₂HPO₄ (50.0 kg), NaH₂PO₄ (42.3 kg) and (E)-2-(2-ethoxyvinyl)-4-methoxynicotinonitrile (3) (120 kg) in dioxane (744 kg) and H₂O (270 kg) was added NBS (62.7 kg) under nitrogen atmosphere. The reaction mixture was stirred at 20-25 °C for 2 hrs.

The reaction was monitored by HPLC until starting material was consumed. The reaction mixture was added 6-chloropyridazin-3-amine (compound B) (48.6 kg). The reaction mixture was degassed with nitrogen for 2 hrs. The reaction mixture was stirred at 70-75 °C for 2 hrs. The reaction mixture was monitored by HPLC. The reaction mixture was dropwise added into H₂O (1440 kg) at 20-25 °C under nitrogen atmosphere to form suspension. The suspension was stirred at 20-25 °C for 1 hr. The suspension was filtered. The filter cake was washed with H₂O (1440 kg) and dried at 55 °C to obtain the titled compound (108 kg, 291 mol, 78% yield, QNMR: 77%) as a brown solid: ¹H NMR (400 MHz, DMSO-d₆) δ 8.85 (d, J=4.8 Hz, 1H), 8.40 (d, J=9.2 Hz, 1H), 8.29 (s, 1H), 7.55 (d, J=8.8 Hz, 1H), 7.44 (d, J=4.4 Hz, 1H), 4.092 (s, 3H).

### tert-Butyl-(S)-(2-(5-(3-(3-cyano-4-methoxypyridin-2-yl)imidazo[1,2-b]pyridazin-6-yl)-2-fluorophenoxy)propyl)carbamate (9)

To a solution of tert-butyl-(S)-(2-(2-fluoro-5-(4,4,5,5-tetramethyl-1,3,2-dioxaborolan-2-yl) phenoxy)propyl) carbamate (5-Boc) (965 kg) in MeOH (164 kg) and H₂O (535 kg) were added K₂CO₃ (535 kg), 2-(6-chloroimidazo[1,2-b]pyridazin-3-yl)-4-methoxynicotinonitrile (8) (108 kg), CuI(11.1 kg) and Pd(PPh₃)₄ (10.1 kg) under nitrogen atmosphere. The reaction mixture was stirred at 70-75 °C for 2 hrs under nitrogen atmosphere. The reaction mixture was monitored by HPLC until starting Compound 8 was completely consumed. The reaction mixture was dropwise added a solution of (2R)-2-amino-3-sulfanyl-propanoic acid (70.5 kg) in H₂O (1080 kg) at 20-25 °C to form a suspension. The suspension was stirred at 20-25 °C for 2 hrs. The suspension was filtered and the filter cake was washed with EtOAc (650 kg). The filtrate was separated and the aqueous phase was extracted with EtOAc (325 kg). To the combined organic phase was added functional silicone (21.6 kg) and the mixture was stirred at 50-55 °C for 2 hrs under nitrogen atmosphere. The mixture was cooled to 20-25 °C and filtered. The filter cake was washed with EtOAc (292 kg). The filtrate was concentrated at 35-40 °C to about 324 L. To the mixture was added MTBE (240 kg) and the mixture again was concentrated at 35-40 °C to about 324 L. The operation was repeated four times. Yellow solid precipitation formed in the mixture. To the mixture was added MTBE (240 kg) and MeCN (54.0 kg) and the resulting mixture was stirred at 20-25 °C for 2 hrs. The mixture was filtered and the filter cake was washed with MTBE (240 kg). The filter cake (crude product 116 kg) was dissolved in EtOAc (1305 kg) and DCM (384 kg). The solution was purified by silica gel column (348 kg), the silica gel column was washed with a solution of EtOAc (4350 kg) and DCM (1280 kg). The column fractions were concentrated at 35-40 °C to about 348 L. The mixture was added MTBE (258 kg) and concentrated at 35-40 °C to about 348 L. The operation was repeated two times. White solid was formed. The mixture was filtered and the filter cake was washed with MTBE (258 kg). The filter cake was dried to obtain the titled compound (62.5 kg, 120 mol, 45% yield, QNMR: 99%) as a white solid: ¹H NMR (400 MHz, DMSO-d₆) δ 8.84 (d, J=6.0 Hz, 1H), 8.33 (d, J=9.6 Hz, 1H), 8.26 (s, 1H), 7.97-8.02 (m, 1H), 7.95-7.96 (m, 1H), 7.66-7.68 (m, 2H), 7.39-7.42 (m, 2H), 7.05-7.07 (m, 1H), 4.59-4.62 (m, 1H), 4.11 (s, 3H), 3.18-3.32 (m, 2H), 1.06-1.33 (m, 12H).

### (S)-2-(6-(3-((1-Aminopropan-2-yl)oxy)-4-fluorophenyl)imidazo[1,2-b]pyridazin-3-yl)-4-methoxynicotinonitrile hydrochloride (10-HCl)

To a solution of tert-butyl (S)-(2-(5-(3-(3-cyano-4-methoxypyridin-2-yl) imidazo [1,2-b]pyridazin-6-yl)-2-fluorophenoxy)propyl)carbamate (9) (62.5 kg) in dry MeOH (989 kg) was added HCl/MeOH (439 kg) under nitrogen atmosphere. The reaction mixture was stirred at 15-20 °C for 15 hrs. The reaction mixture was monitored by HPLC until starting material was consumed. The reaction mixture was filtered. The filter cake was washed with MeOH (148 kg) and dried at 40-45 °C for 20 hrs to obtain the titled compound (52.0 kg, 105 mol, 87% yield) as a white solid: ¹H NMR (400 MHz, DMSO-d₆) δ 8.59 (d, J=6.0 Hz, 1H), 8.27 (s, 1H), 8.18 (d, J=9.6 Hz, 1H), 7.93 (d, J=9.6 Hz, 1H), 7.57 (d, J=7.6 Hz, 1H), 7.34-7.36 (m, 1H), 7.30 (d, J=6.4 Hz, 1H), 7.11-7.13 (m, 1H), 4.62-4.66 (m, 1H), 4.05 (s, 3H), 3.30-3.34 (m, 1H), 3.22-3.25 (m, 1H), 1.26 (d, J=6.0 Hz, 3H).

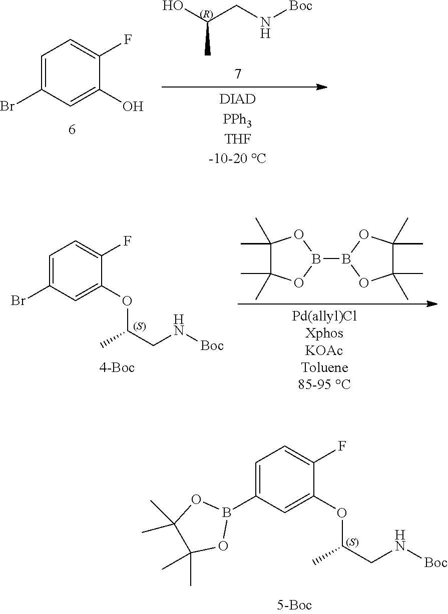

### tert-Butyl (S)-(2-(5-bromo-2-fluorophenoxy)propyl)carbamate (4-Boc)

To a solution of 5-bromo-2-fluorophenol (6) (95.0 kg) in dry THF (507 kg) was added tert-butyl (R)-(2-hydroxypropyl) carbamate (7) (104 kg) and PPh₃ (169 kg) under nitrogen atmosphere. The reaction mixture was dropwise added DIAD (131 kg) at −10 to −5 °C. The reaction mixture was stirred at 20-25 °C for 1 hr under nitrogen atmosphere. The reaction mixture was monitored by HPLC until starting material was consumed. The reaction mixture was added MTBE (351 kg) and H₂O (475 kg) under nitrogen atmosphere. The mixture was stirred at 20-25 °C for 30 mins. The mixture was separated and the aqueous phase was extracted with MTBE (141 kg). The combine organic phase was concentrated at 40-45 °C to about 285 L. The mixture was added n-heptane (195 kg). The operation was repeated two times. The mixture was concentrated at 40-45 °C to about 95.0 L. To the mixture was added n-heptane (650 kg) and the mixture stirred at 20-25 °C for 2 hrs. The mixture was filtered. The filter cake was washed with n-heptane (195 kg). The filtrate was concentrated at 30-35 °C to about 190 L. The mixture was then purified by column chromatography (SiO₂ (285 kg), n-heptane: ethyl acetate=1: 0 to 10: 1). The column fractions were combined and concentrated at 30-35 °C to about 285 L. To the concentrated solution was added toluene (249 kg). This operation was repeated three times. The resulting solution was concentrated at 30-35 °C to obtain the titled compound (414 kg, 416 mol, 83% yield, QNMR: 35%) as a yellow oil: 1H NMR (400 MHz, DMSO-d₆) δ 7.13-7.17 (m, 1H), 7.06-7.08 (m, 1H), 6.81-7.04 (m, 1H), 4.87 (s, 1H), 4.32-4.33 (m, 1H), 3.37-3.40 (m, 1H), 3.13-3.18 (m, 1H), 0.78-1.50 (m, 12H).

### tert-Butyl (S)-(2-(2-fluoro-5-(4,4,5,5-tetramethyl-1,3,2-dioxaborolan-2-yl)phenoxy)propyl)carbamate (5-Boc)

A mixture of Pd(allyl)Cl₂ (381 g) and Xphos (1.29 kg) in toluene (835 kg) was degassed with nitrogen for 2 hrs. The reaction mixture was stirred at 20-25 °C for 2 hrs under nitrogen atmosphere. The reaction mixture was added tert-butyl (S)-(2-(5-bromo-2-fluorophenoxy) propyl) carbamate (4-Boc) (414 kg), 4,4,4′,4′,5,5,5′,5′-octamethyl-2,2′-bi(1,3,2-dioxaborolane) (107 kg) and KOAc (163 kg) under nitrogen atmosphere. The reaction mixture was stirred at 85-90 °C for 30 hrs under nitrogen atmosphere. The reaction mixture was monitored by HPLC until starting material was consumed. The reaction mixture was added H₂O (435 kg) and stir for 30 mins. The reaction mixture was separated and the organic phase was washed with H₂O (290 kg). The organic phase was looped by Cuno (3M™) filter to obtain the titled compound (1000 kg, 379 mol, 90% yield) as a black oil.

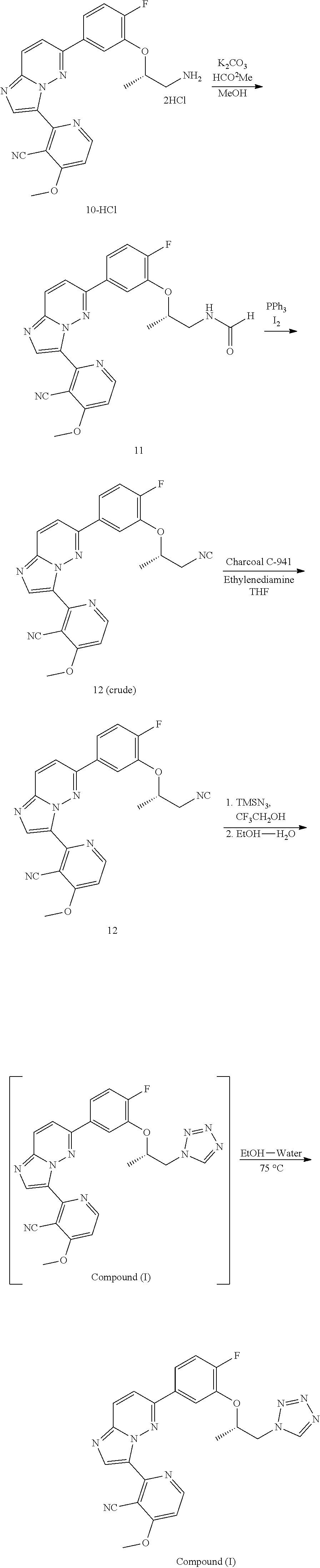

### (S)—N-(2-(5-(3-(3-Cyano-4-methoxypyridin-2-yl)imidazo[1,2-b]pyridazin-6-yl)-2-fluorophenoxy)propyl)formamide (11)

A 200 L glass reactor was assembled with a mechanical stirrer, thermocouple, and a nitrogen inlet, and the apparatus was purged with nitrogen. Methanol (62 L) and potassium carbonate (5653 g) were charged to the reactor and agitated for about 20 min at approximately 24 °C. Compound 10-HCl (6200 g) was then charged to the reactor and agitated for about 20 min at approximately 25 °C. Methyl formate (33.5 L) was charged to the mixture at about 18 °C and the resultant reaction mixture was stirred at approximately 20 °C for about 19 hours until the IPC-1 by HPLC passed. The batch was concentrated under vacuum at about 48 °C to remove most of methanol and methyl formate under reduced pressure.

The distillation residue was diluted slowly with water (52.7 L) at about 27 °C. The reaction mixture was filtered, and the filter cake was washed with water (40.3 L) and MTBE (31 L). The filter cake was dried by pulling vacuum on the filter for 36 hours, then transferred to drying trays and dried under vacuum at about 65 °C until constant weight to afford Compound 11 (5422 g): ¹H NMR (400 MHz, DMSO-d₆) δ 8.85 (d, J=6.0 Hz, 1H), 8.40 (d, J=9.6 Hz, 1H), 8.29-8.31 (m, 1H), 8.26 (s, 1H), 7.96-8.08 (m, 3H), 7.67-7.71 (m, 1H), 7.39-7.44 (m, 2H), 4.55-4.67 (m, 1H), 4.11 (s, 3H), 3.39-3.44 (m, 2H), 1.31 (d, J=6.4 Hz, 3H).

### (S)-2-(6-(4-Fluoro-3-(1-isocyanopropan-2-yl) oxy)phenyl)imidazo[1,2-b]pyridazin-3-yl)-4-methoxynicotinonitrile (Compound 12)

A 100 L glass reactor was assembled with a mechanical stirrer, thermocouple, and a nitrogen inlet, and the apparatus was purged with nitrogen. Compound 11 (5474 g), dichloromethane (DCM, 54.7 L), triethylamine (10.9 L) and triphenylphosphine (8041 g) were charged to the reactor and stirred for 15 min at about 17 °C. Iodine (6224 g) was added slowly in five separate portions at around 20-29 °C. The batch was stirred at about 21 °C for about 2.5 hours until starting material was consumed.

A 5% aqueous sodium bicarbonate (NaHCO₃) solution, prepared by dissolving NaHCO₃ (5479 g) in water (110 L), and half of this volume was charged to the reactor in portions at about 27 °C. The batch was stirred at about 27 °C for about 15 min. The layers were separated, and the bottom organic layer was collected while the upper aqueous layer was kept in the reactor. DCM (27.4 L) was charged to the reactor. The batch was stirred for about 20 minutes, and then the layers were separated. The combined organic layer was extracted with the other half of the 5% aqueous sodium bicarbonate solution (57 L) and stirred for 25 minutes. The layers were then separated, and the organic layer was distilled under reduced pressure at 38 °C until distillation stopped.

The residue was transferred into a 200 L reactor together with DCM (16.4 L). MTBE (54.7 L) was charged into the reactor with stirring and the resultant mixture was stirred at 18 °C for about 3 hours. The batch was filtered, and the filter cake was washed with mixed solvent of DCM-MTBE (27.5 L, 1:4, v/v). The filter cake was dried on the funnel by pulling vacuum through the filter cake for about 25 hours to afford 4975 g of crude Compound 12.

### Purification of Compound 12

A 200 L glass reactor was assembled with a mechanical stirrer, thermocouple, and a nitrogen inlet, and the apparatus was purged with nitrogen. Dichloromethane (DCM, 145 L), crude Compound 12 (9691 g) were charged to the reactor and agitated for 25 min at about 21 °C. Charcoal C-941 (5814 g) and ethylenediamine (4.46 L) were charged to the reactor and agitated for about 4.5 hours at about 19 °C. The batch was filtered through a layer of Celite (4845 g) and the filter cake was washed with DCM (58 L). The combined filtrate and wash were concentrated under reduced pressure at about 40 °C.

The distillation residue was transferred with MTBE (97 L) into a 200 L reactor. The resultant mixture was stirred at about 20 °C for about 3 hours. The batch was filtered and the filter cake washed with MTBE (19 L). The filter cake was dried by pulling nitrogen through the filter for 42 hours to afford purified Compound 12 (9032 g): ¹H NMR (400 MHz, DMSO-d₆) δ 8.84 (d, J=6.0 Hz, 1H), 8.41 (d, J=9.6 Hz, 1H), 8.26 (s, 1H), 8.04 (d, J=9.6 Hz, 1H), 7.96-7.98 (m, 1H), 7.72-7.76 (m, 1H), 7.41-7.48 (m, 2H), 4.84-4.85 (m, 1H), 4.11 (s, 3H), 3.83-3.98 (m, 2H), 1.39 (s, 3H).

### 2-[6-[4-Fluoro-3-[(1S)-1-methyl-2-(tetrazol-1-yl) ethoxy]phenyl]imidazo[1,2-b]pyridazin-3-yl]-4-methoxypyridine-3-carbonitrile (Compound (I))

A 50 L glass reactor was assembled with a mechanical stirrer, thermocouple, additional funnel and a nitrogen inlet, and the apparatus was purged with nitrogen. Trifluoroethanol (6 L) and Compound 12 (1000 g) were charged to the reactor and agitated to form a solution. The solution was heated to about 56 °C. Trimethylsilyl azide (TMSN₃, 0.61 L) was charged to the reactor in small portions over a period of about 1 hours at approximately 65 °C and the reaction mixture was agitated for an additional 8.0 hours at about 56 °C. The batch was cooled to about 17 °C and sodium bicarbonate (392 g) was added. The mixture was stirred at about 20 °C for about 1.5 hours. The batch was filtered, and the filter cake was washed with trifluoroethanol (1 L). The filtrate and wash were combined.

A 50 L glass reactor containing pre-filtered MTBE (20 L) was charged with the combined filtrate and wash. The resultant mixture was stirred at about 21 °C for about 1 hour. The batch was filtered and filter caked washed with MTBE (4 L). The filter cake was dried by pulling nitrogen through the filter for 4 hours to afford crude Compound (I) (976 g).

### Purification of Compound (I)

A 100 L glass reactor was assembled with a mechanical stirrer, thermocouple, and a nitrogen inlet, and the apparatus was purged with nitrogen. DCM (63.4 L) and wet crude Compound (I) (9700 g) were charged to the reactor and stirred for about 20 minutes at 15 °C. The batch was filtered through Celite (2536 g) and charcoal (2536 g) and the filter cake was washed with 38 L of DCM. The resulting mixture was distilled under vacuum at about 40 °C until almost dry. Ethanol (EtOH; 9.5 L) was then added and concentrated under vacuum until dry to afford purified Compound (I) (5765 g): ¹H NMR (400 MHz, DMSO-d₆) δ 9.40 (s, 1H), 8.86 (d, J=6.0 Hz, 1H), 8.41 (d, J=9.6 Hz, 1H), 8.27 (s, 1H), 8.00 (d, J=9.6 Hz, 1H), 7.86-7.88 (m, 1H), 7.68-7.72 (m, 1H), 7.37-7.44 (m, 2H), 5.03-5.05 (m, 1H), 4.82-4.94 (m, 2H), 4.11 (s, 3H), 1.38 (d, J=6.0 Hz, 3H).

### Example 2. Compound (I) Synthesis with Flow Chemistry

The following process was used to synthesize Compound (I) at a 1-kg scale.

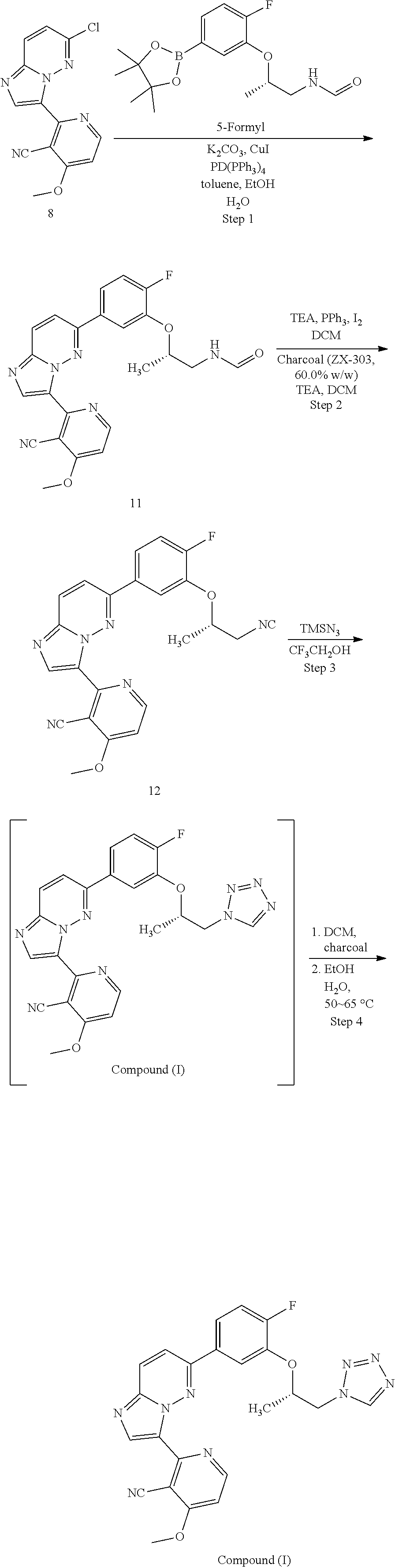

### (S)—N-(2-(5-(3-(3-Cyano-4-methoxypyridin-2-yl)imidazo[1,2-b]pyridazin-6-yl)-2-fluorophenoxy)propyl)formamide (11)

To a solution of 2-(6-chloroimidazo [1,2-b]pyridazin-3-yl)-4-methoxynicotinonitrile (8, 730 g, QNMR: 94%) in toluene (5.79 L), H₂O (4.65 L) and EtOH (1.93 L) were added (S)—N-(2-(2-fluoro-5-(4,4,5,5-tetramethyl-1,3,2-dioxaborolan-2-yl)phenoxy)propyl)formamide (5-Formyl, 1.01 kg, QNMR: 59%), K₂CO₃ (2.32 kg), CuI (91.5 g) and Pd(PPh₃)₄ (22.2 g) at 20 °C under nitrogen protection. The reaction mixture was stirred at 75 °C for 4 hrs and then cooled to 45 °C. Then THF (17.2 L) was added to the reaction mixture at 45 °C and the mixture stirred for 0.5-1 hrs. The reaction mixture was filtered through celite (50% w/w) and the filter cake was washed with THF (2.07 L). The filtrate was separated. The organic phase was isolated. The functional silicone (iMoLboX-LMat-NH21x from Golden Keys High-tech Materials Co., Ltd., Guizhou, China, 40% w/w, 276 g) was added to the organic phase at 45 °C and then stirred for 2 hrs. The mixture was filtered through celite (300% w/w, 2.07 kg) and the filter cake was washed with THF (1.84 L). The functional silicone (iMoLbox-Lmat-NH21x, 40% w/w, 276 g) was added to the filtrate at 45 °C and the mixture stirred for 2 hrs. The mixture was filtered through celite (300% w/w, 2.07 kg) and the filter cake was washed with THF (1.84 L). The filtrate was concentrated under vacuum to 1-2 volumes (V) and swapped with EtOAc to remove residual THF. Then EtOAc (4.82 L) was added to the mixture at 25 °C and stirred for 1 hr to form a suspension. The suspension was filtered and the filter cake was washed with EtOAc (1.37 L). The filter cake was triturated with EtOAc (4.13 L) at 25 °C for 1 hr and then filtered. The filter cake was washed with EtOAc (1.37 L). The filter cake was dissolved in THF (27.6 L) at 25 °C. The solution was purified using a Cuno (3M™) water filter (type R, 120 mL/min, cycle three times, rinse the carbon sheet with THF (1.37 L) at a time). The solution was combined and concentrated under vacuum to 1-2 V then swapped with EtOAc to remove residual THF. Then EtOAc (1.76 L) was added to the mixture at 25 °C and stirred for 1 hr to form a suspension. The suspension was filtered and the filter cake was washed with EtOAc (1.18 L). The filter cake was dried at 55 °C for 10-20 hrs to obtained the titled compound (780 g, QNMR: 96.0%) as a yellow solid: ¹H NMR (400 MHz, DMSO-d₆) δ 8.85 (d, J=6.0 Hz, 1H), 8.40 (d, J=9.6 Hz, 1H), 8.29-8.31 (m, 1H), 8.26 (s, 1H), 7.96-8.08 (m, 3H), 7.67-7.71 (m, 1H), 7.39-7.44 (m, 2H), 4.55-4.67 (m, 1H), 4.11 (s, 3H), 3.39-3.44 (m, 2H), 1.31 (d, J=6.4 Hz, 3H).

### (S)-2-(6-(4-fluoro-3-((1-isocyanopropan-2-yl) oxy)phenyl)imidazo[1,2-b]pyridazin-3-yl)-4-methoxynicotinonitrile (12)

To a solution of (S)—N-(2-(5-(3-(3-cyano-4-methoxypyridin-2-yl)imidazo[1,2-b]pyridazin-6-yl)-2-fluorophenoxy)propyl)formamide (11) (625 g, QNMR: 94.0%) in DCM (5.88 L) was added TEA (852 g) at 25 °C under nitrogen protection. Triphenylphosphine (863 g) was added to the reaction mixture in 10 batches at 25 °C and stirred for 0.5-1 hrs. Then iodine (668 g) was added to the reaction mixture in 5 batches at 20-30 °C and stirred for 1-2 hrs. Aqueous NaHCO₃ (5.0%, 2.94 L) was added dropwise to the reaction mixture at 20 °C and stirred for 0.5-1 hrs. The mixture was separated. The aqueous phase was extracted with DCM (2.94 L). The organic phases were combined and washed with aqueous NaHCO₃ (5.0%, 2.94 L). The organic phase was concentrated under vacuum to 1-2 V. Then DCM (1.76 L) and MTBE (5.89 L) was added to the mixture to form a suspension and stirred at 25 °C for 1-3 hrs. The suspension was filtered, and the filter cake was washed with DCM:MTBE=1: 4 (2.94 L). To a solution of TEA (211 g) in DCM (8.83 L) was added active carbon (ZX-303, 235 g, 60.0% w/w) and stirred at 20 °C for 0.5 hrs. Then the filter cake was added to the mixture and stirred at 20 °C for 4-6 hrs. The mixture was filter with celite (352 g, 50.0% w/w) and washed with DCM (3.53 L). The filtrate was concentrated under vacuum to give the crude product. The crude product was triturated with MTBE (5.89 L) at 25 °C for 1-3 hrs and then filtered. The filter cake was washed with MTBE (1.18 L). The filter cake was dried at 45 °C for 20-26 hrs to obtain the titled compound (459 g, QNMR: 96.0%) as a brown solid: ¹H NMR (400 MHz, DMSO-d₆) δ 8.84 (d, J=6.0 Hz, 1H), 8.41 (d, J=9.6 Hz, 1H), 8.26 (s, 1H), 8.04 (d, J=9.6 Hz, 1H), 7.96-7.98 (m, 1H), 7.72-7.76 (m, 1H), 7.41-7.48 (m, 2H), 4.84-4.85 (m, 1H), 4.11 (s, 3H), 3.83-3.98 (m, 2H), 1.39 (s, 3H).

### 2-[6-[4-fluoro-3-[(1S)-1-methyl-2-(tetrazol-1-yl)ethoxy]phenyl]imidazo[1,2-b]pyridazin-3-yl]-4-methoxy-pyridine-3-carbonitrile (Compound (I))

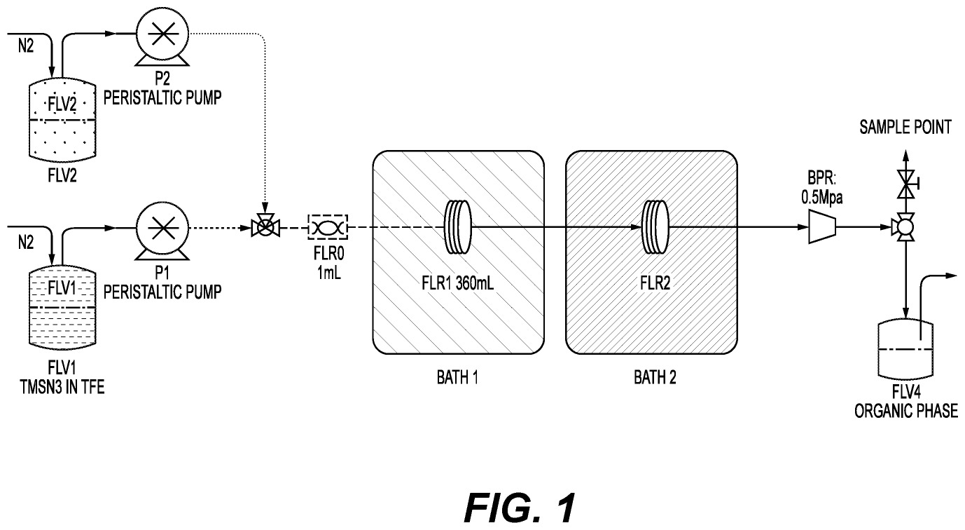

Preparation of Solution A (FLV2): (S)-2-(6-(4-fluoro-3-((1-isocyanopropan-2-yl)oxy)phenyl)imidazo[1,2-b]pyridazin-3-yl)-4-methoxynicotinonitrile (1.00 kg, QNMR: 97.4%) was dissolved in CF₃CH₂OH (5.00 L). Preparation of Solution B (FLV1): TMSN₃ (399 g) was dissolved in CF₃CH₂OH (5.00 L). The equipment was assembled according to the equipment flowchart (FIG. 1). After the equipment passed the leak test, the oil bath temperature was raised to 120 °C. The system flowed with 5.3 mL/min (Solution A) and 5.1 mL/min (Solution B) through a 360 mL coil reactor that was in the oil bath and then a 50.0 mL cooling segment at 25 °C. The reaction system flowed into aq. NaHCO₃ (5.00% wt, 7.00 L) and THF (3.50 L) (FLV4) at 25 °C under nitrogen protection and stirred for 0.5 hrs. The phases were separated. The organic layer was concentrated under vacuum to 1-2 V and then repeatedly swapped with THF to remove residual H₂O until KF<5.5% to obtain a mixture. Then MTBE (20.0 L) was added to the mixture and stirred at 25 °C for 0.5 hrs to form a suspension. The suspension was filtered and the cake was washed with MTBE (4.00 L). The filter cake was concentrated under vacuum to give the crude product as yellow solid. The crude product was dissolved in DCM (10.0 L) at 20 °C. The mixture was filtered through celite (40% w/w, 400 g) and active carbon (40% w/w, 400 g) and the filter cake was washed with DCM (6.00 L). The filter liquor was concentrated under vacuum to 1-2 V and then repeatedly swapped with EtOH to remove residual DCM to obtain a mixture. Then EtOH (2.00 L) and H₂O (4.00 L) were added to the mixture and stirred at 79 °C until almost all the crude product dissolved. Pure titled compound (1.00 g) was added at 74.5 °C as seeds. Water (2.40 L) was added dropwise to the mixture at 73 °C. The mixture was cooled to 70 °C and stirred for 5.5 hrs, then cooled to 24 °C and stirred for 1.5 hrs to form a suspension. The suspension was filtered and washed cake with EtOH: H₂O (1: 1.6 v/v, 3.40 L). The filter cake was dried at 55 °C for 20-30 hrs to obtained the titled compound as white solid (870 g, QNMR: 98.4%): ¹H NMR (400 MHz, DMSO-d₆) δ 9.40 (s, 1H), 8.86 (d, J=6.0 Hz, 1H), 8.41 (d, J=9.6 Hz, 1H), 8.27 (s, 1H), 8.00 (d, J=9.6 Hz, 1H), 7.86-7.88 (m, 1H), 7.68-7.72 (m, 1H), 7.37-7.44 (m, 2H), 5.03-5.05 (m, 1H), 4.82-4.94 (m, 2H), 4.11 (s, 3H), 1.38 (d, J=6.0 Hz, 3H).

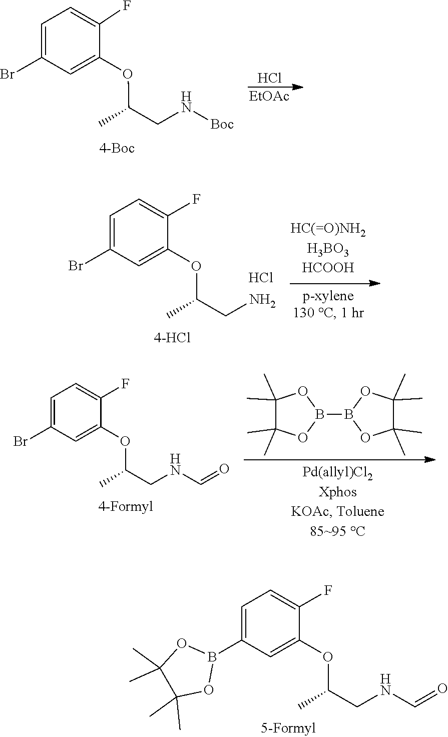

### (S)-2-(5-bromo-2-fluorophenoxy) propan-1-amine hydrochloride (4-HCl)

To a solution of tert-butyl (S)-(2-(5-bromo-2-fluorophenoxy)propyl) carbamate (4-Boc, 3.65 kg) in EtOAc (18.3 L, 5.0 V) was added HCl/EtOAc (18.3 L, 5.0 V) at 20 °C. The reaction mixture was stirred at 20 °C for 12 hrs. The mixture was filtered and the filter cake was washed with EtOAc (11.0 L, 3.0 V). The filtrate was concentrated under vacuum to give the crude product. The crude product was triturated with MTBE:EtOH=1: 1 (51.1 L, 14.0 V) at 25 °C for 1-3 hrs. The mixture was filtered and the filter cake was washed with MTBE:EtOH=1: 1 (7.30 L, 2.0 V). The filter cake was dried at 45 °C for 20-26 hrs to obtain the titled compound (1.67 kg, QNMR: 96.3%) as a white solid: ¹H NMR (400 MHz, DMSO-d₆) 8.27 (s, 3H), 7.56-7.58 (m, 1H), 7.21-7.25 (m, 2H), 4.75-4.81 (m, 1H), 3.02-3.15 (m, 2H), 1.25 (d, J=6.4 Hz, 3H).

### (S)—N-(2-(5-bromo-2-fluorophenoxy)propyl)formamide (4-Formyl)

To a solution of (S)-2-(5-bromo-2-fluorophenoxy) propan-1-amine hydrochloride (4-HCl) (1.43 kg) in p-xylene (14.3 L) was added formamide (243 g), boric acid (15.2 g) and formic acid (609 g) at 25 °C. The reaction mixture was stirred at 125 °C for 0.5-1 hrs, then the reaction mixture was cooled to 15 °C. Aqueous NaHCO₃ (10% w/w, 4.40 L) was added to the reaction mixture and stirred at 15 °C for 10-20 mins. Then EtOAc (4.87 L) was added to the mixture at 25 °C and stirred for 10-20 mins. The mixture was separated and the bottom aqueous layer was extracted with EtOAc (980 mL). The organic phase was combined and washed with aq·NaCl (36.0%, 980 mL). The organic phase was concentrated under vacuum to 1-2 V and then swapped with EtOAc to remove residual p-xylene to give the crude product as oil. The crude product was added EtOAc (139 mL) and heptane (4.13 L) and stirred at 25 °C for 1 hr to form a suspension. The suspension was filtered and washed with EtOAc:heptane=1: 30 (717 mL). The filter cake was dried below 55 °C for 10-20 hrs to obtain the titled compound as a brown solid (1.30 kg): ¹H NMR (400 MHz, DMSO-d₆) δ 8.24 (s, 1H), 8.05 (s, 1H), 7.46-7.49 (m, 1H), 7.13-7.22 (m, 2H), 4.51-4.59 (m, 1H), 3.24-3.32 (m, 1H), 3.34-3.38 (m, 1H), 1.22 (d, J=6.0 Hz, 3H).

### (S)—N-(2-(2-fluoro-5-(4,4,5,5-tetramethyl-1,3,2-dioxaborolan-2-yl)phenoxy)propyl)formamide (5-Formyl)

To a solution of Pd(allyl)Cl₂ (4.22 g) and Xphos (14.3 g) in toluene (9.10 L) was stirred at 25 °C for 1-2 hrs. (S)—N-(2-(5-bromo-2-fluorophenoxy)propyl)formamide (4-Formyl) (1.30 kg), 4,4,4′,4′,5,5,5′,5′-octamethyl-2,2′-bi(1,3,2-dioxaborolane) (1.19 kg) and KOAc (1.81 kg) were added to the mixture at 25 °C. The reaction mixture was stirred at 90 °C for 13-16 hrs. The reaction mixture was cooled to 15 °C, water (3.90 L) and MTBE (3.90 L) were added, and the resulting mixture stirred at 25 °C for 10-20 mins. The mixture was separated and the organic phase was combined and washed with H₂O (2.60 L). The organic phase was concentrated under vacuum to 1-2 V to obtained the titled compound as brown oil (1.81 kg): ¹H NMR (400 MHz, DMSO-d₆) δ 8.23 (s, 1H), 8.06 (s, 1H), 7.35-7.37 (m, 1H), 7.27-7.31 (m, 1H), 7.22-7.25 (m, 1H), 4.46-4.55 (m, 1H), 3.31-3.33 (m, 1H), 3.33-3.35 (m, 1H), 1.29 (s, 12H), 1.21 (d, J=4.0 Hz, 3H).

### Example 3. Flow Synthesis of Compound (I) in One Step from Compound 10

### Process C

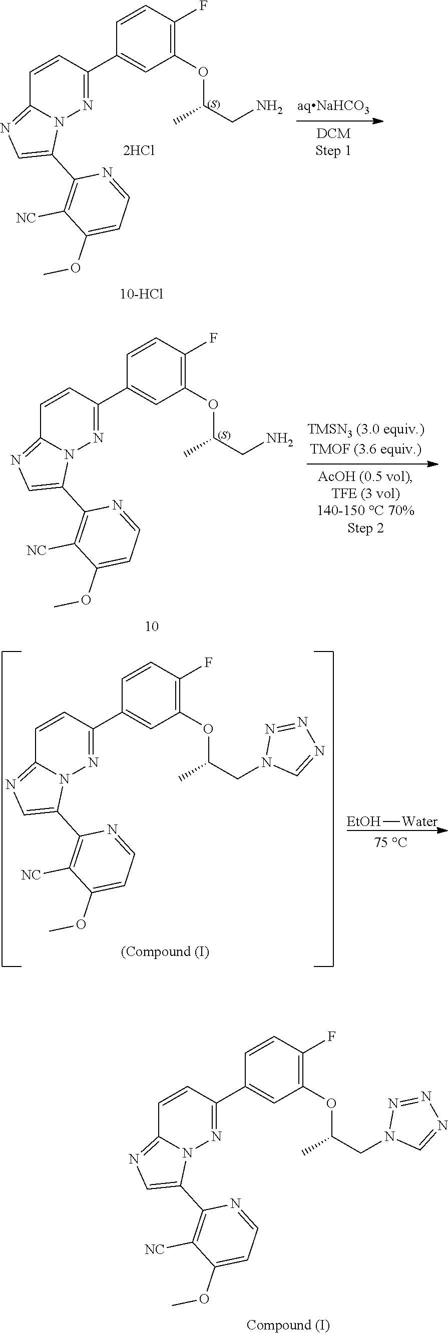

### (S)-2-(6-(3-((1-aminopropan-2-yl) oxy)-4-fluorophenyl) imidazo [1,2-b]pyridazin-3-yl)-4-methoxynicotinonitrile (10)

To a solution of (S)-2-(6-(3-((1-aminopropan-2-yl)oxy)-4-fluorophenyl)imidazo[1,2-b]pyridazin-3-yl)-4-methoxynicotinonitrile hydrochloride (10-HCl, 15.0 g) in DCM (239 g, 12.0 V) and MeOH (23.7 g, 2.00 V) was added aq·NaHCO₃ (5.0% w/w, 15.0 V) to adjust pH γ-8 at 25 °C. The reaction mixture was stirred at 35 °C for 1 hr. The reaction mixture was separated. The aqueous phase was extracted with DCM/MeOH (6/1, 262.7 g, 14.0 V). The combined organic phases were washed with H₂O (150 g, 10.0 V). The organic phase was concentrated under vacuum to 1-2 V below 45 °C and then repeatedly swapped with n-heptane to remove the residual DCM and form a suspension. Then the suspension was stirred at 25 °C for 1-3 hrs. The suspension was filtered and the filter cake was washed with n-heptane (10.3 g, 1.00 V). The filter cake was dried at 40 °C for 4-6 hrs to obtain the titled compound (11.5 g, 89.0% yield) as a white solid: ¹H NMR (400 MHz, DMSO-d₆) δ 8.85 (d, J=6.0 Hz, 1H), 8.40 (d, J=9.6 Hz, 1H), 8.26 (s, 1H), 8.02 (d, J=9.6 Hz, 1H), 7.94-7.97 (m, 1H), 7.60-7.73 (m, 1H), 7.40-7.43 (m, 2H), 4.45-4.50 (m, 1H), 4.11 (s, 3H), 2.74-2.85 (m, 2H), 1.53 (s, 2H), 1.30 (d, J=6.0 Hz, 3H).

### 2-[6-[4-fluoro-3-[(1S)-1-methyl-2-(tetrazol-1-yl)ethoxy]phenyl]imidazo[1,2-b]pyridazin-3-yl]-4-methoxy-pyridine-3-carbonitrile (Compound (I))

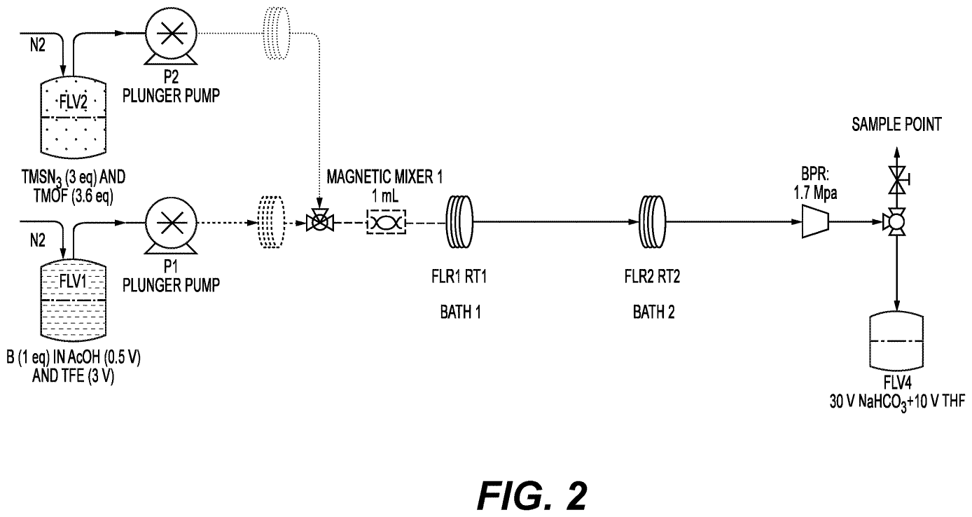

Preparation of Solution A: (S)-2-(6-(3-((1-aminopropan-2-yl) oxy)-4-fluorophenyl)imidazo [1,2-b]pyridazin-3-yl)-4-methoxynicotinonitrile (10) (10.0 g) in CF₃CH₂OH (30.0 mL) was added AcOH (5.0 mL) at 15 °C under nitrogen protection. Preparation of Solution B: TMSN₃ (8.05 g) in TMOF (8.90 g) at 15 °C under nitrogen protection. The equipment was assembled according to the equipment flowchart (FIG. 2). After the equipment passed the leak test, the oil bath temperature was raised to 140 °C. The system flowed with 2.72 mL/min (Solution A) and 1.26 mL/min (Solution B) through a 60 mL coil reactor that is in the oil bath and then a 5 mL cooling segment maintained at 10-20 °C. The reaction mixture flowed into aqueous NaHCO₃ (5.00% wt, 300 mL) and THF (100 mL) at 15 °C under nitrogen protection and stirred at 15 °C for 0.5 hrs. The mixture was separated. The organic layer was concentrated under vacuum to 1-2 volumes (V) and then repeatedly swapped with THF to remove residual H₂O until KF<5.5% to obtain a mixture. Then MTBE (200 mL) was added to the mixture at 15 °C and stirred at 15 °C for 3.0 hrs to form a suspension. The suspension was filtered and washed the cake with MTBE (40.0 mL). The filtrate was concentrated under vacuum to give crude Compound (I) as yellow solid.

To solution of the crude Compound (I) dissolved in DCM (100 mL) was added activated carbon (0.98 g, ZX-303, 10% w/w) at 15 °C under nitrogen protection and stirred for 1 hr. Then the mixture was filtered through celite (40% w/w, 4.00 g) and active carbon (40% w/w, 4.00 g) and the filter cake was washed with DCM (60.0 mL). The filter liquor was concentrated under vacuum to 1-2 V and then swapped with EtOH to remove residual DCM to obtain a mixture.

Ethanol (20.0 mL) and water (40.0 mL) were added to the mixture and this was stirred at 79 °C until almost all the crude product dissolved. Pure titled compound (1.00 mg) was added at 74 °C as seeds. Water (24.0 mL) was dropwise added to the mixture at 74 °C. The mixture was cooled to 70 °C and stirred for 5.5 hrs, and then cooled to 24 °C and stirred for 5 hrs to form a suspension. The suspension was filtered and the cake washed with EtOH: H₂O (1: 1.6 v/v, 34 mL). The filter cake was dissolved in EtOH (40.0 mL) and H₂O (40.0 mL) at 20 °C. The mixture was heated to 79 °C and stirred at 79 °C for 5 hrs. Water (24.0 mL) was dropwise added to the mixture at 74 °C. The mixture was cooled to 70 °C and stirred for 5.5 hrs, and then cooled to 24 °C and stirred for 5 hrs to form a suspension. The suspension was filtered and the cake washed with EtOH: H₂O (1: 1.6 v/v, 34.0 mL). The filter cake was dried at 55 °C for 20-30 hrs to obtain the titled compound as white solid (7.95 g): ¹H NMR (400 MHz, DMSO-d₆) δ 9.40 (s, 1H), 8.86 (d, J=6.0 Hz, 1H), 8.41 (d, J=9.6 Hz, 1H), 8.27 (s, 1H), 8.00 (d, J=9.6 Hz, 1H), 7.86-7.88 (m, 1H), 7.68-7.72 (m, 1H), 7.37-7.44 (m, 2H), 5.03-5.05 (m, 1H), 4.82-4.94 (m, 2H), 4.11 (s, 3H), 1.38 (d, J=6.0 Hz, 3H).

Although the foregoing invention has been described in some detail by way of illustration and Example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A process for preparing Compound (I): 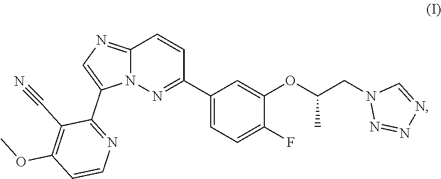 [SMILES: COc1ccnc(-c2cnc3ccc(-c4ccc(F)c(O[C@@H](C)Cn5cnnn5)c4)nn23)c1C#N]
the process comprising:
(a) reacting Compound 10: 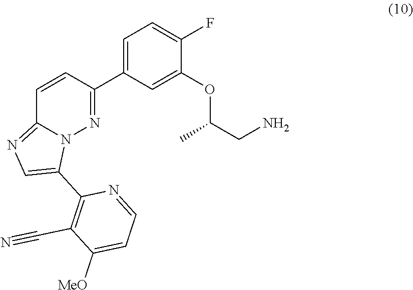 [SMILES: COc1ccnc(-c2cnc3ccc(-c4ccc(F)c(O[C@@H](C)CN)c4)nn23)c1C#N]
and a formylating agent under suitable conditions to prepare Compound 11: 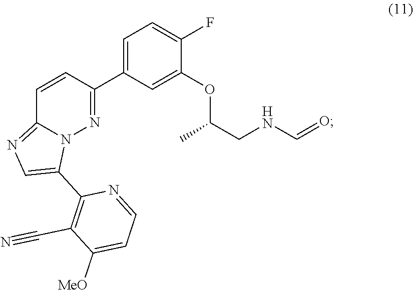 [SMILES: COc1ccnc(-c2cnc3ccc(-c4ccc(F)c(O[C@@H](C)CNC=O)c4)nn23)c1C#N]
(b) reacting Compound 11 and a dehydrating agent under suitable conditions to prepare Compound 12: 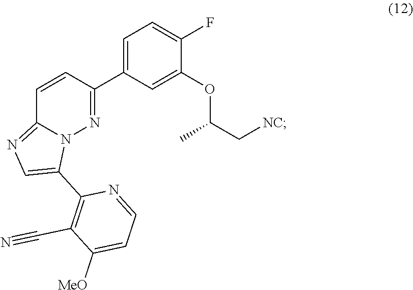
(c) reacting Compound 12 and an azide reagent under suitable conditions to prepare Compound (I).

2. The process of claim 1, wherein the formylating agent comprises trimethyl orthoformate, triethyl orthoformate, methyl formate and a base, chloral, formic acid, acetic formic anhydride, ammonium formate, carbon dioxide and trimethoxysilane, carbon dioxide and phenylsilane, or ethyl bromodifluoroacetate.

3. The process of claim 1, wherein the dehydrating agent is iodine and triphenylphosphine, phosphorus oxychloride and a base, 4-toluenesulfonyl chloride and a base, methanesulfonyl chloride and a base, methanesulfonic anhydride and a base, trifluoromethanesulfonic anhydride and a base, or 1,3-benzenedisulfonyl dichloride and a base.

4. The process of claim 3, wherein the base is triethylamine, diisopropylethylamine, pyridine, N-methylimidazole, DABCO, lithium bicarbonate, lithium carbonate, sodium bicarbonate, sodium carbonate, potassium bicarbonate, or potassium carbonate.

5. The process of claim 1, wherein the azide reagent is a compound of Formula II:
R¹—N₃ (II)
wherein
R¹ is Na, K, tert-butyl, benzyl, o-OMe benzyl, p-OMe benzyl, p-toluenesulfonyl, or 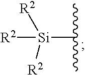
and
each R² is independently C₁₋₆ alkyl or phenyl.

6. The process of claim 5, wherein the azide reagent is trimethylsilyl azide.

7. The process of claim 1, comprising preparing Compound 10 by deprotecting Compound 9: 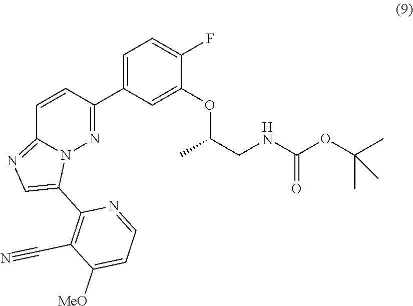 [SMILES: COc1ccnc(-c2cnc3ccc(-c4ccc(F)c(O[C@@H](C)CNC(=O)OC(C)(C)C)c4)nn23)c1C#N]
under suitable conditions to prepare Compound 10.

8. The process of claim 7, comprising preparing Compound 9 by reacting Compound 8: 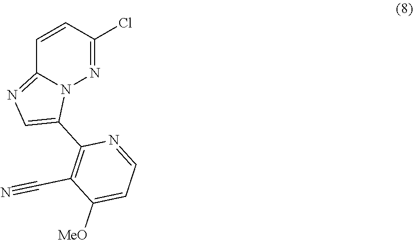 [SMILES: COc1ccnc(-c2cnc3ccc(Cl)nn23)c1C#N]
and
Compound 5-Boc: 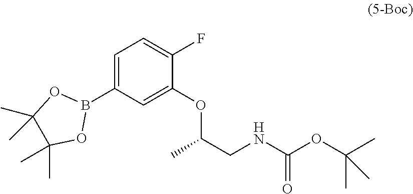 [SMILES: C[C@@H](CNC(=O)OC(C)(C)C)Oc1cc(B2OC(C)(C)C(C)(C)O2)ccc1F]
under suitable conditions to prepare Compound 9.

9. A process for preparing Compound (I): 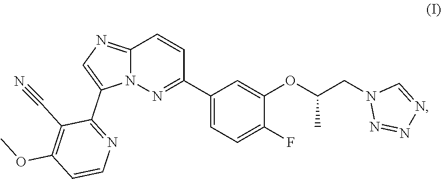 [SMILES: COc1ccnc(-c2cnc3ccc(-c4ccc(F)c(O[C@@H](C)Cn5cnnn5)c4)nn23)c1C#N]
the process comprising:
(a) reacting Compound 8: 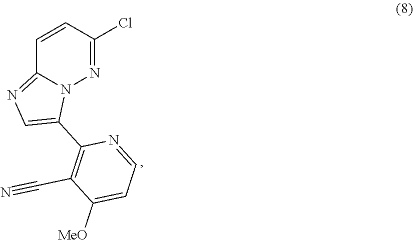 [SMILES: COc1ccnc(-c2cnc3ccc(Cl)nn23)c1C#N]
and Compound 5-Formyl: 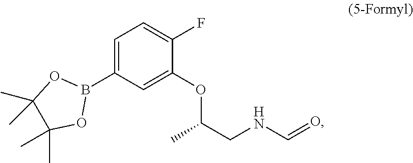 [SMILES: C[C@@H](CNC=O)Oc1cc(B2OC(C)(C)C(C)(C)O2)ccc1F]
under suitable conditions to prepare Compound 11: 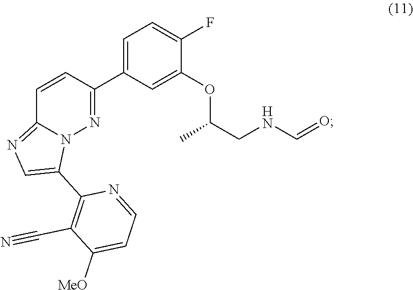 [SMILES: COc1ccnc(-c2cnc3ccc(-c4ccc(F)c(O[C@@H](C)CNC=O)c4)nn23)c1C#N]
(b) reacting Compound 11 and a dehydrating agent under suitable conditions to prepare Compound 12: 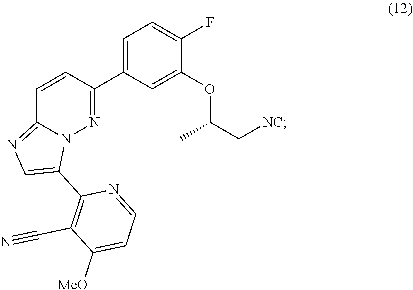
and
(c) reacting Compound 12 and an azide reagent under suitable conditions to prepare Compound (I).

10. The process of claim 9, wherein reacting Compound 8 and Compound 5-Formyl comprises a palladium catalyst.

11. The process of claim 10, wherein the palladium catalyst comprises tetrakis(triphenylphosphine)palladium(0), bis(triphenylphosphine)palladium(II) dichloride, palladium(II) acetate, 1,1′-bis(diphenylphosphino)ferrocene-palladium(II) dichloride, tris(dibenzylideneacetone)dipalladium(O), dichloro[9,9-dimethyl-4,5-bis(diphenylphosphino)xanthene]palladium(II), palladium(II) trifluoroacetate, or bis(acetonitrile)dichloropalladium(II).

12. The process of claim 9, wherein reacting Compound 8 and Compound 5-Formyl comprises a copper catalyst.

13. The process of claim 12, wherein the copper catalyst comprises copper(I) iodide, copper(I) bromide, copper(I) chloride, or copper(I) trifluoromethanesulfonate.

14. The process of claim 9, comprising preparing Compound 5-Formyl by reacting Compound 4-Formyl: 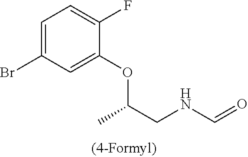
and bis(pinacolato)diboron under suitable conditions to prepare Compound 5-Formyl.

15. A process for preparing Compound (I): 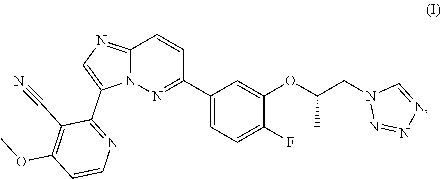 [SMILES: COc1ccnc(-c2cnc3ccc(-c4ccc(F)c(O[C@@H](C)Cn5cnnn5)c4)nn23)c1C#N]
the process comprising reacting Compound 10: 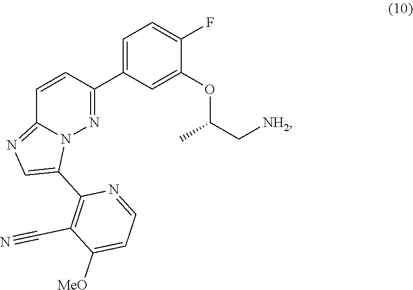 [SMILES: COc1ccnc(-c2cnc3ccc(-c4ccc(F)c(O[C@@H](C)CN)c4)nn23)c1C#N]
an azide reagent, and a formylating agent, under suitable conditions to prepare Compound (I).

16. The process of claim 15, wherein the azide reagent is trimethylsilyl azide.

17. The process of claim 15, wherein the formylating agent comprises trimethyl orthoformate, triethyl orthoformate, methyl formate and a base, chloral, formic acid, acetic formic anhydride, ammonium formate, carbon dioxide and trimethoxysilane, carbon dioxide and phenylsilane, or ethyl bromodifluoroacetate.

18. The process of claim 8, comprising preparing Compound 8: 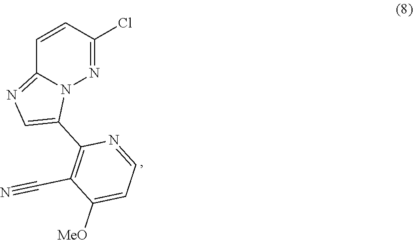 [SMILES: COc1ccnc(-c2cnc3ccc(Cl)nn23)c1C#N]
the process comprising:
(a) reacting Compound 1: 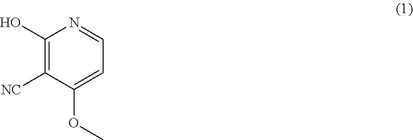
and a chlorinating agent to prepare Compound 2: 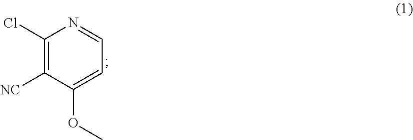
(b) reacting Compound 2 and Compound A: 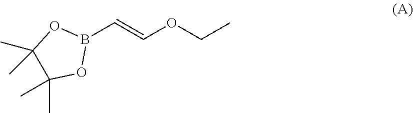
under suitable conditions to prepare Compound 3: 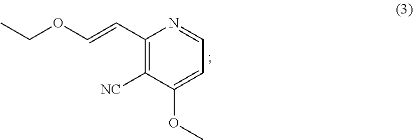
(c) reacting Compound 3, Compound B: 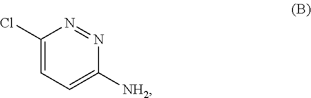
a brominating agent under suitable conditions to prepare Compound 8.

19. A compound having the structure of Compound Ia: 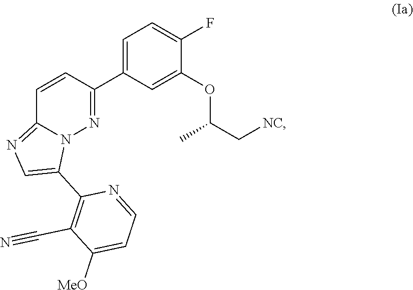
or a salt thereof.

20. A compound of Formula III: 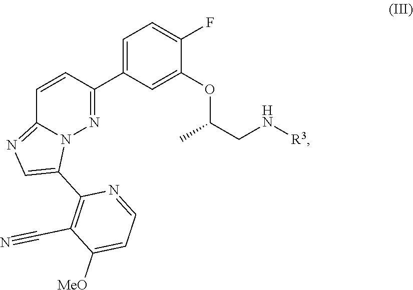
or a salt thereof, wherein
R³ is H,

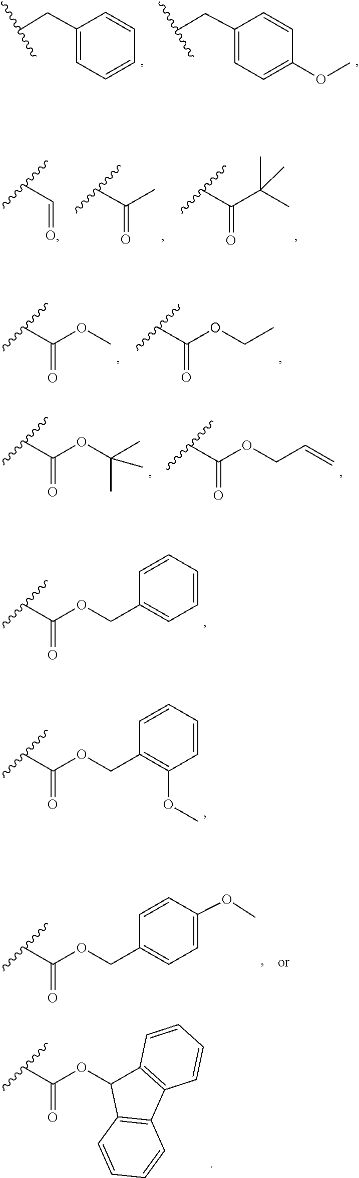

21. The compound of claim 20, having the structure: 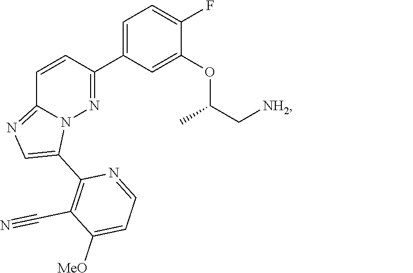 [SMILES: COc1ccnc(-c2cnc3ccc(-c4ccc(F)c(O[C@@H](C)CN)c4)nn23)c1C#N]
or a salt thereof.

22. A compound of Formula IV: 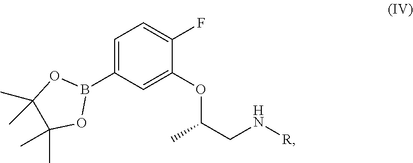
or a salt thereof,
wherein R is

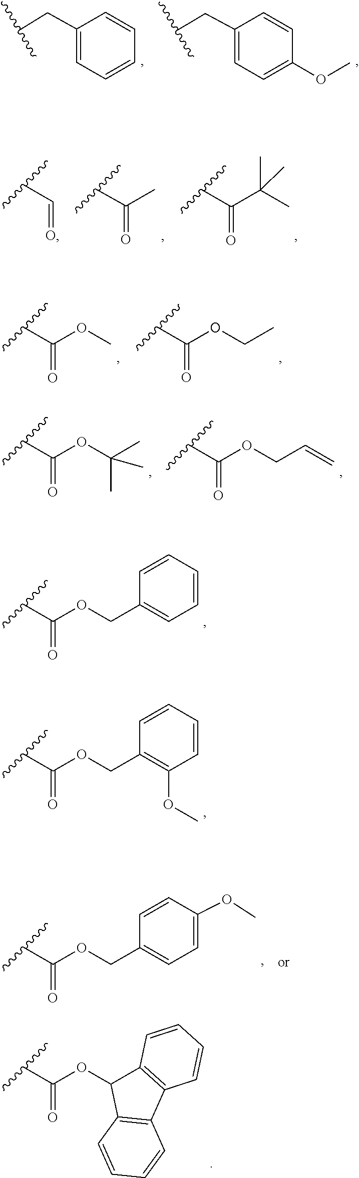

23. A compound of Formula V: 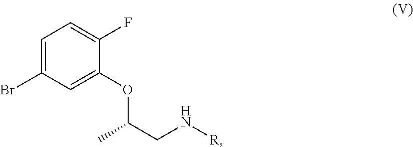
or a salt thereof,
wherein R is [chemical structure]
